(19) United States
(12) Patent Application Publication
(10) Pub. No.: US 2025/0163116 A1
(43) Pub. Date: May 22, 2025

(54) NEW METHOD TO TREAT INFECTIOUS DISEASES

(71) Applicants: INSERM (INSTITUT NATIONAL DE LA SANTÉ ET DE LA RECHERCHE MÉDICALE), Paris (FR); CENTRE NATIONAL DE LA RECHERCHE SCIENTIFIQUE, Paris (FR); UNIVERSITÉ PARIS CITÉ, Paris (FR)

(72) Inventors: Sandrine BOURDOULOUS, Paris (FR); Jason ZIVERI, Paris (FR); Loïc LE GUENNEC, Paris (FR)

(21) Appl. No.: 18/842,489

(22) PCT Filed: Mar. 15, 2023
(86) PCT No.: PCT/EP2023/056642

§ 371 Date: Aug. 29, 2024

(30) Foreign Application Priority Data
Mar. 16, 2022 (EP) 22305312.5

(51) Int. Cl. C07K 14/475; A61K 38/00; A61P 31/04

(52) U.S. Cl. CPC C07K 14/475; A61P 31/04; A61K 38/00

(57) ABSTRACT

The present invention relates to the treatment of infectious diseases. In this study, the inventors identified the potential protective mechanisms involved in the maintenance of cerebrovascular integrity during bacterial infection, the inventors performed RNA analysis of primary endothelial cells isolated from human dermal (HDMECs) or brain (HBMECs) microvessels, which were left uninfected or were infected in vitro with meningococci. They found 40 genes specifically regulated in cerebral endothelial cells upon infection and notably ANGPTL4. They showed that, in the context of sepsis. ANGPTL4 is a major barrier-stabilizing protein conferring protection against bacterial infection. They also demonstrated the potential of ANGPTL4 (or derived peptide) as adjuvant treatment to reduce bacterial-induced vascular dysfunction and mortality. Thus, the present invention relates to peptide derived from the protein ANGPTL4 and their used in the treatment of infectious diseases.

### FIELD OF THE INVENTION

The present invention relates to peptide derived from the protein ANGPTL4 and their used in the treatment of infectious diseases.

### BACKGROUND OF THE INVENTION

Sepsis is one of the most common deadly diseases worldwide, now recognized as a global health priority by the World Health Organization [1]. Every hour, about 1,000 people die from sepsis worldwide, and the number of sepsis cases continues to increase dramatically. It is one of the few conditions to strike with equal ferocity in resource-poor areas and in the developed world. Globally, more than 49 million people are afflicted by sepsis every year, ending to the death of 11 million patients [1, 2]. Patients with these conditions are often treated in a hospital's intensive care unit, with an estimated $24 billion annual cost. The inflation-adjusted aggregate costs for treating patients hospitalized for this condition increased on average annually by 19%. Despite high treatment expenditures, sepsis is often fatal. Those who survive severe sepsis are more likely to have permanent organ damage, cognitive impairment, and physical disability.

Vascular endothelial cells, which are among the first cells in the body that come into contact with circulating bacteria and/or bacterial molecules, are major targets of sepsis-induced events [1-3]. The human vasculature is profoundly altered by the mix of microbial virulence factors and pro-inflammatory mediators released from activated blood cells. Most endothelium physiological functions are disturbed, leading to increased vascular permeability, activation of coagulation, and participation in the inflammatory response. Severe endothelial dysfunction contributes to hypoxic injury to diverse organs, causing multi-organ failure [3]. Disseminated intravascular coagulation is also a major complication of sepsis.

Regimen for patients with sepsis consists of a combination of antibiotic treatment, removal of the source of infection, as well as hemodynamic, respiratory, and metabolic support. Aggressive fluid resuscitation and hemodynamic support are used to restore tissue perfusion and normalize cellular metabolism [2]. However, the mortality rates of severe septic patients or patients in septic shock remain high. So far, multiple clinical trials that aimed at controlling the host inflammatory response and coagulopathy in severe infection have yielded limited clinical success [1]. New strategies are therefore urgently needed to support vascular integrity/barrier function, implement endothelial cytoprotection mechanisms, and limit detrimental haemostatic and inflammatory processes.

The blood brain barrier (BBB), which constitutes a heavily restricting barrier to tightly regulate central nervous system homeostasis, has intrinsic capacity for inflammatory and hemostasis regulation [4, 5]. BBB is formed by continuous non-fenestrated specialized endothelial cells expressing intercellular tight junction proteins, which restrict paracellular diffusion, and specific transporters, which regulate the selective transport and metabolism of substances from blood to brain [6, 7]. The high coverage of pericytes and smooth muscle cells constitute an additional barrier [8]. Finally, a thick basement membrane, in close contact with astrocytic foot processes, also forms a limiting membrane [9]. These specific features contribute to protect the brain from invading pathogens. However, the cytoprotective mechanisms involved in maintenance of BBB integrity during infection remain poorly known.

A paradigm of invasive bacterial pathogen causing severe form of septiciemia and meningitis is Neisseria meningitidis (Nm, meningococcus). Neisseria meningitidis is an extracellular Gram-negative diplococcus restricted to humans, that normally resides in the nasopharynx of approximately 10% of the population, as an asymptomatic carriage. Invasive infections by this bacterium lead to severe septic shock [10]. Once in the blood circulation, Nm adheres to the apical cell surface of endothelial cells, particularly in the microcirculation where the blood flow velocities are slower as compared to large vessels, facilitating bacterial attachment [11]. Interaction of Nm with the human microvasculature is responsible for an endothelial dysfunction syndrome, which, in worst cases, leads to purpura fulminans, a life-threatening syndrome associating septic shock with vascular leakage, extensive thrombosis and death in 30% of the patients [4]. In addition, Nm interacts with brain microvascular endothelial cells. Colonization of brain capillaries, in the subarachnoid space, the parenchyma and the choroid plexus [12], allows bacteria to breach this tight barrier and reach the meninges and cerebrospinal fluid where they replicate, causing meningitis. In contrast to what is observed in periphery, this step occurs with a minimal loss of BBB integrity, with no sign of intra-vascular coagulation or vascular leakage.

### SUMMARY OF THE INVENTION

To identify the potential protective mechanisms involved in the maintenance of cerebrovascular integrity during bacterial infection, the inventors performed RNA analysis of primary endothelial cells isolated from human dermal (HDMECs) or brain (HBMECs) microvessels, which were left uninfected or were infected in vitro with meningococci. They found 40 genes specifically regulated in cerebral endothelial cells upon infection. In consistence with their hypothesis, those include negative regulators of the innate immune response that might confer protection from excessive inflammation, and factors with potential vascular stabilization activity, that might contribute to cerebrovascular protection.

Looking for paracrine and juxtacrine molecules/signalling that may underlie BBB maintenance, they focused their interest on ANGPTL4 (Angiopoietin Like 4), which is a secreted glycoprotein, with pleiotropic role in vascular permeability, angiogenesis, glucose homeostasis, lipid metabolism and inflammation [13]. ANGPTL4 is a member of the angiopoietin-like protein family (ANGPTL 1-8) that share a similar structure with a N-terminal coiled-coil domain and C-terminal fibrinogen-like domain separated by a cleavable linker. In particular, ANGPTL4 shares high sequence homology and related functions with ANGPTL3 and ANGPTL8. Long considered as an orphan ligand because it does not bind to angiopoietin tyrosine kinase receptors Tie1 and Tie2, recent studies indicate that ANGPTL4 has many binding partners, such as lipoprotein lipase, integrins, cadherins or syndecans [13]. To date, its role in vascular integrity remains unclear. Known to be induced by hypoxia and produced by vascular cells in ischemic pathologies, ANGPTL4 was shown to exert both angiogenic and anti-angiogenic effects [14, 15], and to increase or reduce vascular permeability depending on the pathophysiological conditions, tissue contexts and/or its binding partner, release, and proteolysis [16-20].

The inventors showed in this study that, in the context of sepsis, ANGPTL4 is a major barrier-stabilizing protein conferring protection against bacterial infection. They also demonstrated the potential of ANGPTL4 (or derived peptide) as adjuvant treatment to reduce bacterial-induced vascular dysfunction and mortality.

Thus, the present invention relates to peptide derived from the protein ANGPTL4 and their used in the treatment of infectious diseases. Particularly, the invention is defined by its claims.

### DETAILED DESCRIPTION OF THE INVENTION

### Peptides or Proteins of the Invention

A first aspect of the invention relates to a peptide derived from the protein ANGPTL4 comprising or consisting of the amino acid sequence X₁SALERRLSACGSX₁₄X₁₅ (SEQ ID NO: 65) or a function-conservative variant thereof, wherein X₁ is no amino acid or leucine (L), X₁₄ is no amino acid or alanine (A) and X₁₅ is no amino acid or cysteine (C).

In particular embodiment, X₁ is leucine (L), X₁₄ is no amino acid and X₁₅ is no amino acid (Peptide P12).

In particular embodiment, X₁ is leucine (L), X₁₄ is alanine (A) and X₁₅ is cysteine (C). (Peptide P9).

In particular embodiment, X₁ is no amino acid, X₁₄ is alanine (A) and X₁₅ is cysteine (C) (Peptide P13).

In particular embodiment, X₁ is leucine (L), X₁₄ is alanine (A) and X₁₅ is no amino acid).

In particular embodiment, the peptide derived from the protein ANGPTL4 comprises or consist of the amino acid residue at position 66 to the amino acid residue at position 78 in SEQ ID NO: 1 wherein the amino acids at position 76 are any naturally occurring amino acid (peptide P12 or peptide P12′) or a function-conservative variant thereof.

In particular embodiment, the peptide comprises or consist of the amino acid sequence LSALERRLSACGS (SEQ ID NO:55) (Peptide 12).

In particular embodiment, the peptide derived from the protein ANGPTL4 comprises or consist of the amino acid residue at position 67 to the amino acid residue at position 80 in SEQ ID NO: 1 wherein the amino acids at position 76 and 80 are any naturally occurring amino acid (peptide P13 or peptide P13′) or a function-conservative variant thereof.

In particular embodiment, the peptide comprises or consist of the amino acid sequence SALERRLSACGSAC (SEQ ID NO:58) (Peptide 13).

In particular embodiment, the peptide derived from the protein ANGPTL4 comprises or consist of the amino acid residue at position 66 to the amino acid residue at position 80 in SEQ ID NO: 1 wherein the amino acids at position 76 and 80 are any naturally occurring amino acid (peptide P9 or peptide P9′) or a function-conservative variant thereof.

Thus, a second aspect of the invention relates to a peptide derived from the protein ANGPTL4 comprising or consisting of the amino acid residue at position 66 to the amino acid residue at position 80 in SEQ ID NO:1 wherein the amino acids at position 76 and 80 are any naturally occurring amino acid (peptide P9 or peptide P9′) or a function-conservative variant thereof.

In other word, a first aspect of the invention relates to a peptide derived from the protein ANGPTL4 comprising or consisting of the amino acids sequence LSALERRLSAX¹¹GSAX¹⁵ (SEQ ID NO:2, peptide P9 or peptide P9′) wherein X¹¹ and X¹⁵ are any naturally occurring amino acid or a function-conservative variant thereof.

In a particular embodiment and according to all peptides or proteins of the invention, the amino acids at position 76 and 80 can be amino acids selected from the group consisting of cysteine (Cys or C) or alanine (Ala or A) or serine (Ser or S).

Thus, in a particular embodiment, the invention relates to a peptide derived from the protein ANGPTL4 comprising or consisting of the amino acid residue at position 66 to the amino acid residue at position 80 in SEQ ID NO: 1 wherein the amino acids at position 76 and 80 are amino acids selected from the group consisting of cysteine (Cys or C) or alanine (Ala or A), or serine (Ser or S) or a function-conservative variant thereof.

In other word, the invention relates to a peptide derived from the protein ANGPTL4 comprising or consisting of the amino acids sequence LSALERRLSAX¹¹GSAX¹⁵ (SEQ ID NO: 2) wherein X¹¹ and X¹⁵ are amino acids selected from the group consisting of cysteine (Cys or C) or alanine (Ala or A), or serine (Ser or S) or a function-conservative variant thereof.

As used herein, the term “ANGPTL4” for “Angiopoietin-like 4” denotes a serum hormone directly that regulate lipid and glucose metabolism. The native full length ANGPTL4 can form higher order structures via intermolecular disulfide bonds. The N-terminal region of ANGPTL4 (nANGPTL4) is responsible for its assembly. The full length ANGPTL4 undergoes proteolytic cleavage at the linker region, releasing nANGPTL4 and the monomeric C-terminal portion of ANGPTL4 (cANGPTL4). For the gene sequence, its Entrez Gene reference number is: 51129; for the protein, the UniProt reference number is: Q9BY76.

Amino acid sequence of the human ANGPTL4 (SEQ
ID NO: 1):
MSGAPTAGAALMLCAATAVLLSAQGGPVQSKSPRFASWDEMNVLAHGLL
QLGQGLREHAERTRSQLSALERRLSACGSACQGTEGSTDLPLAPESRVD
PEVLHSLQTQLKAQNSRIQQLFHKVAQQQRHLEKQHLRIQHLQSQFGLL
DHKHLDHEVAKPARRKRLPEMAQPVDPAHNVSRLHRLPRDCQELFQVGE
RQSGLFEIQPQGSPPFLVNCKMTSDGGWTVIQRRHDGSVDFNRPWEAYK
AGFGDPHGEFWLGLEKVHSITGDRNSRLAVQLRDWDGNAELLQFSVHLG
GEDTAYSLQLTAPVAGQLGATTVPPSGLSVPFSTWDQDHDLRRDKNCAK
SLSGGWWFGTCSHSNLNGQYFRSIPQQRQKLKKGIFWKTWRGRYYPLQA
TTMLIQPMAAEAAS

In another particular embodiment, the invention relates to a peptide derived from the protein ANGPTL4 comprising or consisting of the amino acid residue at position 65 to the amino acid residue at position 80 in SEQ ID NO:1 wherein the amino acids at position 76 and 80 are any naturally occurring amino acid (peptide P6 or peptide P6′) or a function-conservative variant thereof.

In other word and in a particular embodiment, the invention relates to a peptide derived from the protein ANGPTL4 comprising or consisting in the amino acids sequence: QLSALERRLSAX¹²GSAX¹⁶ (SEQ ID NO:3, peptide P6 or peptide P6′) wherein X¹² and X¹⁶ are any naturally occurring amino acid or a function-conservative variant thereof.

In another particular embodiment, the invention relates to a peptide derived from the protein ANGPTL4 comprising or consisting of the amino acid residue at position 65 to the amino acid residue at position 80 in SEQ ID NO:1 wherein the amino acids at position 76 and 80 are amino acids selected from the group consisting of cysteine (Cys or C) or alanine (Ala or A) or serine (Ser or S) or a function-conservative variant thereof.

In other word and in a particular embodiment, the invention relates to a peptide derived from the protein ANGPTL4 comprising or consisting in the amino acids sequence: QLSALERRLSAX¹²GSAX¹⁶ (SEQ ID NO:3) wherein X¹² and X¹⁶ are amino acids selected from the group consisting of cysteine (Cys or C) or alanine (Ala or A), or serine (Ser or S) or a function-conservative variant thereof.

In another particular embodiment, the invention relates to a peptide derived from the protein ANGPTL4 comprising or consisting of the amino acid residue at position 56 to the amino acid residue at position 83 in SEQ ID NO: 1 wherein the amino acids at position 76 and 80 are any naturally occurring amino acid (peptide P4 or peptide P4′) or a function-conservative variant thereof.

In other word and in another particular embodiment, the invention relates to a peptide derived from the protein ANGPTL4 comprising or consisting in the amino acids sequence: REHAERTRSQLSALERRLSAX²¹GSAX²⁵QGT (SEQ ID NO:4, peptide P4 or peptide P4′) wherein X¹² and X¹⁶ are any naturally occurring amino acid or a function-conservative variant thereof.

In another particular embodiment, the invention relates to a peptide derived from the protein ANGPTL4 comprising or consisting of the amino acid residue at position 56 to the amino acid residue at position 83 in SEQ ID NO: 1 wherein the amino acids at position 76 and 80 are amino acids selected from the group consisting of cysteine (Cys or C) or alanine (Ala or A), or serine (Ser or S) or a function-conservative variant thereof.

In other word and in another particular embodiment, the invention relates to a peptide derived from the protein ANGPTL4 comprising or consisting in the amino acids sequence: REHAERTRSQLSALERRLSAX²¹GSAX²⁵QGT (SEQ ID NO:4) wherein X²¹ and X²⁵ are amino acids selected in the group consisting in cysteine (Cys or C) or alanine (Ala or A) or serine (Ser or S) or a function-conservative variant thereof.

In another particular embodiment, the invention relates to a peptide derived from the protein ANGPTL4 comprising or consisting of the amino acid residue at position 38 to the amino acid residue at position 83 in SEQ ID NO: 1 wherein the amino acids at position 76 and 80 are any naturally occurring amino acid (peptide P1 or peptide P1′) or a function-conservative variant thereof.

In other word and in another particular embodiment, the invention relates to a peptide derived from ANGPTL4 comprising or consisting in the amino acids sequence: WDEMNVLAHGLLQLGQGLREHAERTRSQLSALERRLSAX³⁹GSAX⁴³QGT (SEQ ID NO: 5, peptide P1 or peptide P1′) wherein X³⁹ and X⁴³ are any naturally occurring amino acid or a function-conservative variant thereof.

In another particular embodiment, the invention relates to a peptide derived from the protein ANGPTL4 comprising or consisting of the amino acid residue at position 38 to the amino acid residue at position 83 in SEQ ID NO:1 wherein the amino acids at position 76 and 80 are amino acids selected from the group consisting of cysteine (Cys or C) or alanine (Ala or A) or serine (Ser or S) or a function-conservative variant thereof.

In other word and in another particular embodiment, the invention relates to a peptide derived from ANGPTL4 comprising or consisting in the amino acids sequence: WDEMNVLAHGLLQLGQGLREHAERTRSQLSALERRLSAX³⁹GSAX⁴³QGT (SEQ ID NO: 5) wherein X³⁹ and X⁴³ are amino acids selected from the group consisting of cysteine (Cys or C) or alanine (Ala or A) or serine (Ser or S) or a function-conservative variant thereof.

In another particular embodiment, the invention relates to a peptide derived from the protein ANGPTL4 comprising or consisting of the amino acid residue at position 26 to the amino acid residue at position 164 in SEQ ID NO: 1 wherein the amino acids at position 76 and 80 are any naturally occurring amino acid (nANGPTL4 or nANGPTL4′) or a function-conservative variant thereof.

In other word and in another particular embodiment, the invention relates to a peptide derived from ANGPTL4 comprising or consisting in the amino acids sequence: GPVQSKSPRFASWDEMNVLAHGLLQLGQGLREHAERTRSQLSALERRLSAX⁵¹GSAX⁵⁵SQGTEGSTDLPLAPESRVDPEVLHSLQTQLKAQNSRIQQLFHKVAQQQRHLEKQHLRI QHLQSQFGLLDHKHLDHEVAKPARRK (SEQ ID NO:6, nANGPTL4 or nANGPTL4′) wherein X⁵¹ and X⁵⁵ are any naturally occurring amino acid or a function-conservative variant thereof.

In another particular embodiment, the invention relates to a peptide derived from the protein ANGPTL4 comprising or consisting of the amino acid residue at position 26 to the amino acid residue at position 164 in SEQ ID NO: 1 wherein the amino acids at position 76 and 80 are amino acids selected from the group consisting of cysteine (Cys or C) or alanine (Ala or A) or serine (Ser or S) or a function-conservative variant thereof.

In other word and in another particular embodiment, the invention relates to a peptide derived from ANGPTL4 comprising or consisting in the amino acids sequence: GPVQSKSPRFASWDEMNVLAHGLLQLGQGLREHAERTRSQLSALERRLSAX⁵¹GSAX⁵⁵SQGTEGSTDLPLAPESRVDPEVLHSLQTQLKAQNSRIQQLFHKVAQQQRHLEKQHLRI QHLQSQFGLLDHKHLDHEVAKPARRK (SEQ ID NO:6) wherein X⁵¹ and X⁵⁵ are amino acids selected in the group consisting in cysteine (Cys or C) or alanine (Ala or A) or serine (Ser or S) or a function-conservative variant thereof.

In another particular embodiment, the invention relates to a protein derived from the protein ANGPTL4 comprising or consisting of the amino acid residue at position 1 to the amino acid residue at position 406 in SEQ ID NO: 1 wherein the amino acids at position 76 and 80 are any naturally occurring amino acid (ANGPTL4 or ANGPTL4′) or a function-conservative variant thereof.

In other word and in another particular embodiment, the invention relates to a peptide derived from ANGPTL4 comprising or consisting in the amino acids sequence: MSGAPTAGAALMLCAATAVLLSAQGGPVQSKSPRFASWDEMNVLAHGLLQLGQGL REHAERTRSQLSALERRLSAX⁷⁶GSAX⁸⁰QGTEGSTDLPLAPESRVDPEVLHSLQTQLKA QNSRIQQLFHKVAQQQRHLEK QHLRIQHLQSQFGLLDHKHLDHEVAKPARRKRLPE MAQPVDPAHNVSRLHRLPRDCQELFQVGERQSGLFEIQPQGSPPFLVNCKMTSDGG WTVIQRRHDGSVDFNRPWEAYKAGFGDPHGEFWLGLEKVHSITGDRNSRLAVQLRD WDGNAELLQFSVHLGGEDTAYSLQLTAPVAGQLGATTVPPSGLSVPFSTWDQDHDL RRDKNCAKSLSGGWWFGTCSHSNLNGQYFRSIPQQRQKLKKGIFWKTWRGRYYPLQ ATTMLIQPMAAEAAS (SEQ ID NO:7, ANGPTL4 or ANGPTL4′) wherein X⁷⁶ and X⁸⁰ are any naturally occurring amino acid or a function-conservative variant thereof.

In another particular embodiment, the invention relates to a protein derived from the protein ANGPTL4 comprising or consisting of the amino acid residue at position 1 to the amino acid residue at position 406 in SEQ ID NO: 1 wherein the amino acids at position 76 and 80 are amino acids selected from the group consisting of cysteine (Cys or C) or alanine (Ala or A) or serine (Ser or S) or a function-conservative variant thereof.

In other word and in another particular embodiment, the invention relates to a peptide derived from ANGPTL4 comprising or consisting in the amino acids sequence: MSGAPTAGAALMLCAATAVLLSAQGGPVQSKSPRFASWDEMNVLAHGLLQLGQGL REHAERTRSQLSALERRLSAX⁷⁶GSAX⁸⁰QGTEGSTDLPLAPESRVDPEVLHSLQTQLKA QNSRIQQLFHK VAQQQRHLEKQHLRIQHLQSQFGLLDHKHLDHEVAKPARRKRLPE MAQPVDPAHNVSRLHRLPRDCQELFQVGERQSGLFEIQPQGSPPFLVNCKMTSDGG WTVIQRRHDGSVDFNRPWEAYKAGFGDPHGEFWLGLEKVHSITGDRNSRLAVQLRD WDGNAELLQFSVHLGGEDTAYSLQLTAPVAGQLGATTVPPSGLSVPFSTWDQDHDL RRDKNCAKSLSGGWWFGTCSHSNLNGQYFRSIPQQRQKLKKGIFWKTWRGRYYPLQ ATTMLIQPMAAEAAS (SEQ ID NO:7) wherein X⁷⁶ and X⁸⁰ are amino acids selected from the group consisting of cysteine (Cys or C) or alanine (Ala or A) or serine(S) a function-conservative variant thereof.

In a particular embodiment, the peptides or proteins of the invention comprise or consist in the amino acids sequence as set forth in SEQ ID NO: 1 and 8 to 30 and 37 to 64.

Peptide P9:
(SEQ ID NO: 8)
LSALERRLSACGSAC
Peptide P9 C76A, C80A:
(SEQ ID NO: 9)
LSALERRLSAAGSAA
Peptide P9 C76A:
(SEQ ID NO: 10)
LSALERRLSAAGSAC
Peptide P9 C80A:
(SEQ ID NO: 11)
LSALERRLSACGSAA
Peptide P9 C76S, C80S:
(SEQ ID NO: 37)
LSALERRLSASGSAS
Peptide P9 C76S:
(SEQ ID NO: 38)
LSALERRLSASGSAC
Peptide P9 C80S:
(SEQ ID NO: 39)
LSALERRLSACGSAS
Peptide P6:
(SEQ ID NO: 12)
QLSALERRLSACGSAC
Peptide P6 C76A, C80A:
(SEQ ID NO: 13)
QLSALERRLSAAGSAA
Peptide P6: C76A:
(SEQ ID NO: 14)
QLSALERRLSAAGSAC
Peptide P6 C80A:
(SEQ ID NO: 15)
QLSALERRLSACGSAA
Peptide P6 C76S, C80S:
(SEQ ID NO: 40)
QLSALERRLSASGSAS
Peptide P6 C76S:
(SEQ ID NO: 41)
QLSALERRLSASGSAC
Peptide P6 C80S:
(SEQ ID NO: 42)
QLSALERRLSACGSAS
Peptide P4:
(SEQ ID NO: 16)
REHAERTRSQLSALERRLSACGSACQGT
Peptide P4 C76A, C80A:
(SEQ ID NO: 17)
REHAERTRSQLSALERRLSAAGSAAQGT
Peptide P4 C76A:
(SEQ ID NO: 18)
REHAERTRSQLSALERRLSAAGSACQGT
Peptide P4 C80A:
(SEQ ID NO: 19)
REHAERTRSQLSALERRLSACGSAAQGT
Peptide P4 C76S, C80S:
(SEQ ID NO: 43)
REHAERTRSQLSALERRLSASGSASQGT
Peptide P4 C76S:
(SEQ ID NO: 44)
REHAERTRSQLSALERRLSASGSACQGT
Peptide P4 C80S:
(SEQ ID NO: 45)
REHAERTRSQLSALERRLSACGSASQGT
Peptide P1:
(SEQ ID NO: 20)
WDEMNVLAHGLLQLGQGLREHAERTRSQLSALERRLSACGSACQGT
Peptide P1 C76A, C80A:
(SEQ ID NO: 21)
WDEMNVLAHGLLQLGQGLREHAERTRSQLSALERRLSAAGSAAQGT
Peptide P1 C76A:
(SEQ ID NO: 22)
WDEMNVLAHGLLQLGQGLREHAERTRSQLSALERRLSAAGSACQGT
Peptide P1 C80A:
(SEQ ID NO: 23)
WDEMNVLAHGLLQLGQGLREHAERTRSQLSALERRLSACGSAAQGT
Peptide P1 C76S, C80S:
(SEQ ID NO: 46)
WDEMNVLAHGLLQLGQGLREHAERTRSQLSALERRLSASGSASQGT
Peptide P1 C76S:
(SEQ ID NO: 47)
WDEMNVLAHGLLQLGQGLREHAERTRSQLSALERRLSASGSACQGT
Peptide P1 C80S:
(SEQ ID NO: 48)
WDEMNVLAHGLLQLGQGLREHAERTRSQLSALERRLSACGSASQGT
Peptide nANGPTL4:
(SEQ ID NO: 24)
GPVQSKSPRFASWDEMNVLAHGLLQLGQGLREHAERTRSQLSALERRLS
ACGSACQGTEGSTDLPLAPESRVDPEVLHSLQTQLKAQNSRIQQLFHKV
AQQQRHLEKQHLRIQHLQSQFGLLDHKHLDHEVAKPARRK
Peptide nANGPTL4 C76A, C80A:
(SEQ ID NO: 25)
GPVQSKSPRFASWDEMNVLAHGLLQLGQGLREHAERTRSQLSALERRLS
AAGSAAQGTEGSTDLPLAPESRVDPEVLHSLQTQLKAQNSRIQQLFHKV
AQQQRHLEKQHLRIQHLQSQFGLLDHKHLDHEVAKPARRK
Peptide nANGPTL4 C76A:
(SEQ ID NO: 26)
GPVQSKSPRFASWDEMNVLAHGLLQLGQGLREHAERTRSQLSALERRLS
AAGSACQGTEGSTDLPLAPESRVDPEVLHSLQTQLKAQNSRIQQLFHKV
AQQQRHLEKQHLRIQHLQSQFGLLDHKHLDHEVAKPARRK
Peptide nANGPTL4 C80A:
(SEQ ID NO: 27)
GPVQSKSPRFASWDEMNVLAHGLLQLGQGLREHAERTRSQLSALERRLS
ACGSAAQGTEGSTDLPLAPESRVDPEVLHSLQTQLKAQNSRIQQLFHKV
AQQQRHLEKQHLRIQHLQSQFGLLDHKHLDHEVAKPARRK
Peptide nANGPTL4 C76S, C80S:
(SEQ ID NO: 49)
GPVQSKSPRFASWDEMNVLAHGLLQLGQGLREHAERTRSQLSALERRLS
ASGSASQGTEGSTDLPLAPESRVDPEVLHSLQTQLKAQNSRIQQLFHKV
AQQQRHLEKQHLRIQHLQSQFGLLDHKHLDHEVAKPARRK
Peptide nANGPTL4 C76S:
(SEQ ID NO: 50)
GPVQSKSPRFASWDEMNVLAHGLLQLGQGLREHAERTRSQLSALERRLS
ASGSACQGTEGSTDLPLAPESRVDPEVLHSLQTQLKAQNSRIQQLFHKV
AQQQRHLEKQHLRIQHLQSQFGLLDHKHLDHEVAKPARRK
Peptide nANGPTL4 C80S:
(SEQ ID NO: 51)
GPVQSKSPRFASWDEMNVLAHGLLQLGQGLREHAERTRSQLSALERRLS
ACGSASQGTEGSTDLPLAPESRVDPEVLHSLQTQLKAQNSRIQQLFHKV
AQQQRHLEKQHLRIQHLQSQFGLLDHKHLDHEVAKPARRK
Protein ANGPTL4 C76A, C80A:
(SEQ ID NO: 28)
MSGAPTAGAALMLCAATAVLLSAQGGPVQSKSPRFASWDEMNVLAHGLL
QLGQGLREHAERTRSQLSALERRLSAAGSAAQGTEGSTDLPLAPESRVD
PEVLHSLQTQLKAQNSRIQQLFHKVAQQQRHLEKQHLRIQHLQSQFGLL
DHKHLDHEVAKPARRKRLPEMAQPVDPAHNVSRLHRLPRDCQELFQVGE
RQSGLFEIQPQGSPPFLVNCKMTSDGGWTVIQRRHDGSVDFNRPWEAYK
AGFGDPHGEFWLGLEKVHSITGDRNSRLAVQLRDWDGNAELLQFSVHLG
GEDTAYSLQLTAPVAGQLGATTVPPSGLSVPFSTWDQDHDLRRDKNCAK
SLSGGWWFGTCSHSNLNGQYFRSIPQQRQKLKKGIFWKTWRGRYYPLQA
TTMLIQPMAAEAAS
Protein ANGPTL4 C76A:
(SEQ ID NO: 29)
MSGAPTAGAALMLCAATAVLLSAQGGPVQSKSPRFASWDEMNVLAHGLL
QLGQGLREHAERTRSQLSALERRLSAAGSACQGTEGSTDLPLAPESRVD
PEVLHSLQTQLKAQNSRIQQLFHKVAQQQRHLEKQHLRIQHLQSQFGLL
DHKHLDHEVAKPARRKRLPEMAQPVDPAHNVSRLHRLPRDCQELFQVGE
RQSGLFEIQPQGSPPFLVNCKMTSDGGWTVIQRRHDGSVDFNRPWEAYK
AGFGDPHGEFWLGLEKVHSITGDRNSRLAVQLRDWDGNAELLQFSVHLG
GEDTAYSLQLTAPVAGQLGATTVPPSGLSVPFSTWDQDHDLRRDKNCAK
SLSGGWWFGTCSHSNLNGQYFRSIPQQRQKLKKGIFWKTWRGRYYPLQA
TTMLIQPMAAEAAS
Protein ANGPTL4 C80A:
(SEQ ID NO: 30)
MSGAPTAGAALMLCAATAVLLSAQGGPVQSKSPRFASWDEMNVLAHGLL
QLGQGLREHAERTRSQLSALERRLSACGSAAQGTEGSTDLPLAPESRVD
PEVLHSLQTQLKAQNSRIQQLFHKVAQQQRHLEKQHLRIQHLQSQFGLL
DHKHLDHEVAKPARRKRLPEMAQPVDPAHNVSRLHRLPRDCQELFQVGE
RQSGLFEIQPQGSPPFLVNCKMTSDGGWTVIQRRHDGSVDFNRPWEAYK
AGFGDPHGEFWLGLEKVHSITGDRNSRLAVQLRDWDGNAELLQFSVHLG
GEDTAYSLQLTAPVAGQLGATTVPPSGLSVPFSTWDQDHDLRRDKNCAK
SLSGGWWFGTCSHSNLNGQYFRSIPQQRQKLKKGIFWKTWRGRYYPLQA
TTMLIQPMAAEAAS
Protein ANGPTL4 C76S, C80S:
(SEQ ID NO: 52)
MSGAPTAGAALMLCAATAVLLSAQGGPVQSKSPRFASWDEMNVLAHGLL
QLGQGLREHAERTRSQLSALERRLSASGSASQGTEGSTDLPLAPESRVD
PEVLHSLQTQLKAQNSRIQQLFHKVAQQQRHLEKQHLRIQHLQSQFGLL
DHKHLDHEVAKPARRKRLPEMAQPVDPAHNVSRLHRLPRDCQELFQVGE
RQSGLFEIQPQGSPPFLVNCKMTSDGGWTVIQRRHDGSVDFNRPWEAYK
AGFGDPHGEFWLGLEKVHSITGDRNSRLAVQLRDWDGNAELLQFSVHLG
GEDTAYSLQLTAPVAGQLGATTVPPSGLSVPFSTWDQDHDLRRDKNCAK
SLSGGWWFGTCSHSNLNGQYFRSIPQQRQKLKKGIFWKTWRGRYYPLQA
TTMLIQPMAAEAAS
Protein ANGPTL4 C76S:
(SEQ ID NO: 53)
MSGAPTAGAALMLCAATAVLLSAQGGPVQSKSPRFASWDEMNVLAHGLL
QLGQGLREHAERTRSQLSALERRLSASGSACQGTEGSTDLPLAPESRVD
PEVLHSLQTQLKAQNSRIQQLFHKVAQQQRHLEKQHLRIQHLQSQFGLL
DHKHLDHEVAKPARRKRLPEMAQPVDPAHNVSRLHRLPRDCQELFQVGE
RQSGLFEIQPQGSPPFLVNCKMTSDGGWTVIQRRHDGSVDFNRPWEAYK
AGFGDPHGEFWLGLEKVHSITGDRNSRLAVQLRDWDGNAELLQFSVHLG
GEDTAYSLQLTAPVAGQLGATTVPPSGLSVPFSTWDQDHDLRRDKNCAK
SLSGGWWFGTCSHSNLNGQYFRSIPQQRQKLKKGIFWKTWRGRYYPLQA
TTMLIQPMAAEAAS
Protein ANGPTL4 C80S:
(SEQ ID NO: 54)
MSGAPTAGAALMLCAATAVLLSAQGGPVQSKSPRFASWDEMNVLAHGLL
QLGQGLREHAERTRSQLSALERRLSACGSASQGTEGSTDLPLAPESRVD
PEVLHSLQTQLKAQNSRIQQLFHKVAQQQRHLEKQHLRIQHLQSQFGLL
DHKHLDHEVAKPARRKRLPEMAQPVDPAHNVSRLHRLPRDCQELFQVGE
RQSGLFEIQPQGSPPFLVNCKMTSDGGWTVIQRRHDGSVDFNRPWEAYK
AGFGDPHGEFWLGLEKVHSITGDRNSRLAVQLRDWDGNAELLQFSVHLG
GEDTAYSLQLTAPVAGQLGATTVPPSGLSVPFSTWDQDHDLRRDKNCAK
SLSGGWWFGTCSHSNLNGQYFRSIPQQRQKLKKGIFWKTWRGRYYPLQA
TTMLIQPMAAEAAS
Peptide 12:
(SEQ ID NO: 55)
LSALERRLSACGS
Peptide 12 C76A:
(SEQ ID NO: 56)
LSALERRLSAAGS
Peptide 12 C76S:
(SEQ ID NO: 57)
LSALERRLSASGS
Peptide 13:
(SEQ ID NO: 58)
SALERRLSACGSAC
Peptide 13 C76A, C80A:
(SEQ ID NO: 59)
SALERRLSAAGSAA
Peptide 13 C76A:
(SEQ ID NO: 60)
SALERRLSAAGSAC
Peptide 13 C80A:
(SEQ ID NO: 61)
SALERRLSACGSAA
Peptide 13 C76S, C80S:
(SEQ ID NO: 62)
SALERRLSASGSAS
Peptide 13 C76S:
(SEQ ID NO: 63)
SALERRLSASGSAC
Peptide 13 C80S:
(SEQ ID NO: 64)
SALERRLSACGSAS

According to the invention, the peptides or proteins of the invention can also have the mutation S67R and/or R72L.

As used herein, the term “function-conservative variants” refers to those in which a given amino acid residue in a protein or enzyme has been changed (inserted, deleted or substituted) without altering the overall conformation and function of the polypeptide. Such variants include protein having amino acid alterations such as deletions, insertions and/or substitutions. A “deletion” refers to the absence of one or more amino acids in the protein. An “insertion” refers to the addition of one or more of amino acids in the protein. A “substitution” refers to the replacement of one or more amino acids by another amino acid residue in the protein. Typically, a given amino acid is replaced by an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, particularly at least 75%, more particularly at least 85%, still particularly at least 90%, and even more particularly at least 95%, and which has the same or substantially similar properties or functions as the native or parent protein to which it is compared. Two amino acid sequences are “substantially homologous” or “substantially similar” when greater than 80%, particularly greater than 85%, particularly greater than 90% of the amino acids are identical, or greater than about 90%, particularly greater than 95%, are similar (functionally identical) over the whole length of the shorter sequence. Particularly, the similar or homologous sequences are identified by alignment using, for example, the GCG (Genetics Computer Group, Program Manual for the GCG Package, Version 7, Madison, Wisconsin) pileup program, or any of sequence comparison algorithms such as BLAST, FASTA, etc.

In a particular embodiment, the peptides of the invention may have an amino acid sequence having less than 150, 149, 148, 147, 146, 145, 144, 143, 142, 141, 140, 139, 138, 137, 136, 135, 134, 133, 132, 131, 130, 129, 128, 127, 126, 125, 124, 123, 122, 121, 120, 119, 118, 117, 116, 115, 114, 113, 112, 111, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14 or 13 amino acids.

In a particular embodiment, the peptide of the invention comprises at least 70% identity over the peptides or proteins described in the invention (SEQ ID NO: 1 to 30 and 37 to 64 and particularly 1 and 8 to 30 and 37 to 64), even more particularly at least 80%, 81%, 82%, 83%, 84%, 85%, 86%, 87%, 88%, 89%, 90%, 91%, 92%, 93%, 94%, 95%, 96%, 97%, 98%, 99% and is still able to be efficiently reduce the incidence of vascular alteration, intravascular coagulation in bacterial diseases and reduce the heavy toll of sepsis.

Typically, the invention encompasses peptides or proteins comprising or consisting of an amino acid sequence as set forth in SEQ ID NO: 1 to 30 and 37 to 64 and particularly 1 and 8 to 30 and 37 to 64 in which one or more residues have been conservatively substituted with a functionally similar residue and which displays the functional aspects of the peptides or proteins of the invention, i.e. being still able to reduce the incidence of vascular alteration, intravascular coagulation in bacterial diseases and reduce the heavy toll of sepsis.

Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid or another.

The term “conservative substitution” also includes the use of a chemically derivatized residue in place of a non-derivatized residue. “Chemical derivative” refers to a patient peptide having one or more residues chemically derivatized by reaction of a functional side group. Examples of such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Chemical derivatives also include peptides which contain one or more naturally-occurring amino acid derivatives of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine may be substituted for lysine.

In one embodiment, peptides or proteins of the invention may comprise a tag. A tag is an epitope-containing sequence which can be useful for the purification of the peptides. It is attached to by a variety of techniques such as affinity chromatography, for the localization of said peptide or polypeptide within a cell or a tissue sample using immunolabeling techniques, the detection of said peptide or polypeptide by immunoblotting etc. Examples of tags commonly employed in the art are the GST (glutathion-S-transferase)-tag, the FLAG™-tag, the Strep-tag™, V5 tag, myc tag, His tag etc.

In one embodiment, peptides or proteins of the invention may be labelled by a fluorescent dye. Dye-labelled fluorescent peptides are important tools in cellular studies. Peptides can be labelled on the N-terminal side or on the C-terminal side.

### N-Terminal Peptide Labeling Using Amine-Reactive Fluorescent Dyes:

Amine-reactive fluorescent probes are widely used to modify peptides or proteins at the N-terminal or lysine residue. A number of fluorescent amino-reactive dyes have been developed to label various peptides, and the resultant conjugates are widely used in biological applications. Three major classes of amine-reactive fluorescent reagents are currently used to label peptides: succinimidyl esters (SE), isothiocyanates and sulfonyl chlorides.

### C-Terminal Labeling Using Amine-Containing Fluorescent Dyes:

Amine-containing dyes are used to modify peptides using water-soluble carbodiimides (such as EDC) to convert the carboxy groups of the peptides into amide groups. Either NHS or NHSS may be used to improve the coupling efficiency of EDC-mediated protein-carboxylic acid conjugations.

In specific embodiments, it is contemplated that peptides or proteins used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomerular filtration (e.g., less than 45 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes (see e.g., technologies of established by VectraMed, Plainsboro, N.J.). Such linkers may be used in modifying the peptides-derived described herein for therapeutic delivery.

In a particular embodiment, the peptides of the invention can be peptidomimectics. As used herein, the term “peptidomimetic” refers to a polypeptide designed to mimic a peptide such as the peptides of the invention. The term “peptidomimetic” also means a non-peptide chemical moiety. Peptides are short chains of amino acid monomers linked by peptide (amide) bonds, the covalent chemical bonds formed when the carboxyl group of one amino acid reacts with the amino group of another. The shortest peptides are dipeptides, consisting of 2 amino acids joined by a single peptide bond, followed by tripeptides, tetrapeptides, etc. A peptidomimetic chemical moiety can include non-amino acid chemical moieties. A peptidomimetic chemical moiety may also include one or more amino acid that are separated by one or more non-amino acid chemical units. A peptidomimetic chemical moiety does not contain in any portion of its chemical structure two or more adjacent amino acids that are linked by peptide bonds. The term “amino acid” as used herein means glycine, alanine, valine, leucine, isoleucine, phenylalanine, proline, serine, threonine, tyrosine, cysteine, methionine, lysine, arginine, histidine, tryptophan, aspartic acid, glutamic acid, asparagine, glutamine or citrulline.

According to the invention, peptides or proteins may be produced by conventional automated peptide/protein synthesis methods or by recombinant expression. General principles for designing and making peptides/proteins are well known to those of skill in the art.

Peptides or proteins of the invention may be synthesized in solution or on a solid support in accordance with conventional techniques. Various automatic synthesizers are commercially available and can be used in accordance with known protocols as described in Stewart and Young; Tam et al., 1983; Merrifield, 1986 and Barany and Merrifield, Gross and Meienhofer, 1979. Peptides of the invention may also be synthesized by solid-phase technology employing an exemplary peptide synthesizer such as a Model 433A from Applied Biosystems Inc. The purity of any given protein; generated through automated peptide synthesis or through recombinant methods may be determined using reverse phase HPLC analysis. Chemical authenticity of each peptide may be established by any method well known to those of skill in the art.

As an alternative to automated peptide/protein synthesis, recombinant DNA technology may be employed wherein a nucleotide sequence which encodes a protein of choice is inserted into an expression vector, transformed or transfected into an appropriate host cell and cultivated under conditions suitable for expression as described herein below. Recombinant methods are especially preferred for producing longer polypeptides.

A variety of expression vector/host systems may be utilized to contain and express the peptide or protein coding sequence. These include but are not limited to microorganisms such as bacteria transformed with recombinant bacteriophage, plasmid or cosmid DNA expression vectors; yeast transformed with yeast expression vectors (Giga-Hama et al., 1999); insect cell systems infected with virus expression vectors (e.g., baculovirus, see Ghosh et al., 2002); plant cell systems transfected with virus expression vectors (e.g., cauliflower mosaic virus, CaMV; tobacco mosaic virus, TMV) or transformed with bacterial expression vectors (e.g., Ti or pBR322 plasmid; see e.g., Babe et al., 2000); or animal cell systems. Those of skill in the art are aware of various techniques for optimizing mammalian expression of proteins, see e.g., Kaufman, 2000; Colosimo et al., 2000. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Exemplary protocols for the recombinant expression of the peptide substrates or fusion polypeptides in bacteria, yeast and other invertebrates are known to those of skill in the art and a briefly described herein below. U.S. Pat. Nos. 6,569,645; 6,043,344; 6,074,849; and 6,579,520 provide specific examples for the recombinant production of peptides and these patents are expressly incorporated herein by reference for those teachings. Mammalian host systems for the expression of recombinant proteins also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein.

In the recombinant production of the peptides or proteins of the invention, it would be necessary to employ vectors comprising polynucleotide molecules for encoding the peptides-derived. Methods of preparing such vectors as well as producing host cells transformed with such vectors are well known to those skilled in the art. The polynucleotide molecules used in such an endeavor may be joined to a vector, which generally includes a selectable marker and an origin of replication, for propagation in a host. These elements of the expression constructs are well known to those of skill in the art. Generally, the expression vectors include DNA encoding the given protein being operably linked to suitable transcriptional or translational regulatory sequences, such as those derived from a mammalian, microbial, viral, or insect genes. Examples of regulatory sequences include transcriptional promoters, operators, or enhancers, mRNA ribosomal binding sites, and appropriate sequences which control transcription and translation.

The terms “expression vector,” “expression construct” or “expression cassette” are used interchangeably throughout this specification and are meant to include any type of genetic construct containing a nucleic acid coding for a gene product in which part or all of the nucleic acid encoding sequence is capable of being transcribed.

The choice of a suitable expression vector for expression of the peptides or polypeptides of the invention will of course depend upon the specific host cell to be used, and is within the skill of the ordinary artisan. Methods for the construction of mammalian expression vectors are disclosed, for example, in Okayama and Berg, 1983; Cosman et al., 1986; Cosman et al., 1984; EP-A-0367566; and WO 91/18982. Other considerations for producing expression vectors are detailed in e.g., Makrides et al., 1999; Kost et al., 1999. Wurm et al., 1999 is incorporated herein as teaching factors for consideration in the large-scale transient expression in mammalian cells for recombinant protein production.

Expression requires that appropriate signals be provided in the vectors, such as enhancers/promoters from both viral and mammalian sources that may be used to drive expression of the nucleic acids of interest in host cells. Usually, the nucleic acid being expressed is under transcriptional control of a promoter. A “promoter” refers to a DNA sequence recognized by the synthetic machinery of the cell, or introduced synthetic machinery, required to initiate the specific transcription of a gene. Nucleotide sequences are operably linked when the regulatory sequence functionally relates to the DNA encoding the peptide of interest (i.e., 4N1K, a variant and the like). Thus, a promoter nucleotide sequence is operably linked to a given DNA sequence if the promoter nucleotide sequence directs the transcription of the sequence.

Similarly, the phrase “under transcriptional control” means that the promoter is in the correct location and orientation in relation to the nucleic acid to control RNA polymerase initiation and expression of the gene. Any promoter that will drive the expression of the nucleic acid may be used. The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter. Common promoters include, e.g., the human cytomegalovirus (CMV) immediate early gene promoter, the SV40 early promoter, the Rous sarcoma virus long terminal repeat, [beta]-actin, rat insulin promoter, the phosphoglycerol kinase promoter and glyceraldehyde-3-phosphate dehydrogenase promoter, all of which are promoters well known and readily available to those of skill in the art, can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient to produce a recoverable yield of protein of interest. By employing a promoter with well-known properties, the level and pattern of expression of the protein of interest following transfection or transformation can be optimized. Inducible promoters also may be used.

Another regulatory element that is used in protein expression is an enhancer. These are genetic elements that increase transcription from a promoter located at a distant position on the same molecule of DNA. Where an expression construct employs a cDNA insert, one will typically desire to include a polyadenylation signal sequence to effect proper polyadenylation of the gene transcript. Any polyadenylation signal sequence recognized by cells of the selected transgenic animal species is suitable for the practice of the invention, such as human or bovine growth hormone and SV40 polyadenylation signals.

In one embodiment, the peptides or proteins of the invention is linked with at least one cell penetrating peptide.

The terms “cell penetrating peptide” or “CPP” are used interchangeably and refer to cationic cell penetrating peptides, also called transport peptides, carrier peptides, or peptide transduction domains. The CPP, as shown herein, have the capability of inducing cell penetration of a peptide fused to the CPP within 30%, 40%, 50%, 60%, 70%, 80%, 90%, or 100% of cells of a given cell culture population, including all integers in between, and allow macromolecular translocation within multiple tissues in vivo upon systemic administration. A cell-penetrating peptide may also refer to a peptide which, when brought into contact with a cell under appropriate conditions, passes from the external environment in the intracellular environment, including the cytoplasm, organelles such as mitochondria, or the nucleus of the cell, in conditions significantly greater than passive diffusion. Such penetrating peptides may be those described in Fonseca S. B. et al., Advanced Drug Delivery Reviews, 2009, 61:953-964, Johansson et al., Methods in Molecular Biology, 2011, Vol. 683, Chapter 17, in WO2004/011595 and in WO2003/011898.

In one embodiment the CPP is selected in the group consisting in but not limited to Tat peptide, polyarginines peptide, HA2-R9 peptide, Penetratin peptide, Transportan peptide, Vectocell® peptide, maurocalcine peptide, decalysine peptide, HIV-Tat derived PTD4 peptide, Hepatitis B virus Translocation Motif (PTM) peptide, mPrP1-28 peptide, POD, pVEC, EB1, Rath, CADY, Histatin 5, Antp peptide, Cyt86-101 peptide.

To verify whether the peptides or proteins of the invention are still able reduce the incidence of vascular alteration, intravascular coagulation in bacterial diseases and reduce the heavy toll of sepsis, a test may be performed with each peptide. For example, the skilled person can test the ability of the peptides or proteins to protect the integrity of primary human dermal microvascular cells (HDMECs) from meningococcal infection in vitro, and/or to prevent the formation of vascular lesions, thrombosis and the progression to organ failure in vivo using a lipopolysaccharide (LPS)-induced endotoxemia and/or an in vivo model of meningococcal infection.

### Nucleic Acids, Vectors, Recombinant Host Cells

A third aspect of the invention relates to a nucleic acid sequence encoding a peptide or protein (like ANGPTL4) according to the invention.

A fourth aspect of the invention relates to an expression vector comprising a nucleic acid sequence encoding a peptide or protein (ANGPTL4) according to the invention.

According to the invention, expression vectors suitable for use in the invention may comprise at least one expression control element operationally linked to the nucleic acid sequence. The expression control elements are inserted in the vector to control and regulate the expression of the nucleic acid sequence. Examples of expression control elements include, but are not limited to, lac system, operator and promoter regions of phage lambda, yeast promoters and promoters derived from polyoma, adenovirus, retrovirus, lentivirus or SV40. Additional preferred or required operational elements include, but are not limited to, leader sequence, termination codons, polyadenylation signals and any other sequences necessary or preferred for the appropriate transcription and subsequent translation of the nucleic acid sequence in the host system. It will be understood by one skilled in the art that the correct combination of required or preferred expression control elements will depend on the host system chosen. It will further be understood that the expression vector should contain additional elements necessary for the transfer and subsequent replication of the expression vector containing the nucleic acid sequence in the host system. Examples of such elements include, but are not limited to, origins of replication and selectable markers. It will further be understood by one skilled in the art that such vectors are easily constructed using conventional methods or commercially available.

A fifth aspect the invention is a host cell comprising an expression vector as described here above.

According to the invention, examples of host cells that may be used are eukaryote cells, such as animal, plant, insect and yeast cells and prokaryotes cells, such as E. coli. The means by which the vector carrying the gene may be introduced into the cells include, but are not limited to, microinjection, electroporation, transduction, or transfection using DEAE-dextran, lipofection, calcium phosphate or other procedures known to one skilled in the art.

In a particular embodiment, eukaryotic expression vectors that function in eukaryotic cells are used. Examples of such vectors include, but are not limited to, viral vectors such as retrovirus, adenovirus, adeno-associated virus, herpes virus, vaccinia virus, poxvirus, poliovirus; lentivirus, bacterial expression vectors, plasmids, such as pcDNA3 or the baculovirus transfer vectors. Preferred eukaryotic cell lines include, but are not limited to, COS cells, CHO cells, HeLa cells, NIH/3T3 cells, 293 cells (ATCC #CRL1573), T2 cells, dendritic cells, or monocytes.

### Use of the Peptides or Proteins of the Invention

A sixth aspect of the invention relates to a peptide, a protein, nucleic acids, vectors or host cells according to the invention for use in the treatment of an infectious disease in a subject in need thereof.

According to the invention, the infectious disease can be due to a pathogen like a bacterium, a virus, a protozoan, a prion, a viroid, or a fungus. Particularly, infection promoted by any pathogen which induces vascular alterations could be treated with the peptides or proteins of the invention.

Particularly, the protozoan can be the Plasmodium falciparum.

Particularly, the virus can be an influenza virus, such as the Influenza A virus (IAV) or the Influenza B virus (IAB), adenovirus, metapneumovirus, cytomegalovirus, parainfluenza virus (e.g., hPIV-1, hPIV-2, hPIV-3, hPIV-4), the human rhinovirus (HRV), the Human respiratory syncytial virus (HRSV) or a coronavirus.

As used herein, the term “coronavirus” has its general meaning in the art and refers to any member of the Coronaviridae family. Coronavirus is a virus whose genome is plus-stranded RNA of about 27 kb to about 33 kb in length depending on the particular virus. The virion RNA has a cap at the 5′ end and a poly A tail at the 3′ end. The length of the RNA makes coronaviruses the largest of the RNA virus genomes. In particular, coronavirus RNAs encode: (1) an RNA-dependent RNA polymerase; (2) N-protein; (3) three envelope glycoproteins; plus (4) three non-structural proteins. In particular, the coronavirus particle comprises at least the four canonical structural proteins E (envelope protein), M (membrane protein), N (nucleocapsid protein), and S (spike protein). The S protein is cleaved into 3 chains: Spike protein S1, Spike protein S2 and Spike protein S2′. Production of the replicase proteins is initiated by the translation of ORF1a and ORF1ab via a −1 ribosomal frame-shifting mechanism. This mechanism produces two large viral polyproteins, pp1a and pp1ab, that are further processed by two virally encoded cysteine proteases, the papain-like protease (PLpro) and a 3C-like protease (3CLpro), which is sometimes referred to as main protease (Mpro). Coronaviruses infect a variety of mammals and birds. They cause respiratory infections (common), enteric infections (mostly in infants >12 mo.), and possibly neurological syndromes. Coronaviruses are transmitted by aerosols of respiratory secretions. Coronaviruses are exemplified by, but not limited to, human enteric coV (ATCC accession #VR-1475), human coV 229E (ATCC accession #VR-740), human coV OC43 (ATCC accession #VR-920), Middle East respiratory syndrome-related coronavirus (MERS-Cov) and SARS-coronavirus (Center for Disease Control), in particular SARS-Cov1 and SARS-Cov2.

According to the invention, the coronavirus can be a MERS-COV, SARS-COV, SARS-CoV-2 or any new future family members.

Particularly, the pathogen can be a bacterium. Thus, in this case, the invention relates to a peptide, a protein, nucleic acids, vectors or host cells according to the invention for use in the treatment of a bacterial infection (or bacterial disease) in a subject in need thereof.

The inventors notably showed (see examples parts) that a bacterial infection, notably an infection by Neisseria meningitidis, induces vascular dysfunction (or vascular insult or alteration). They showed that peptides or proteins of the invention (ANGPTL4 or peptides or proteins derived from ANGPTL4) exert a protective effect on the integrity of endothelial cell monolayers infected with bacteria (notably N. meningitidis), preserve the integrity of the endothelial cell junctions, prevent the occurrence of vascular alteration, thrombosis and inflammation and the progression to organ failure and death during bacterial infection (like meningococcaemia) and thus can be used as a major vascular stabilization factor conferring protection to bacterial infection and sepsis due to infection like bacterial infection.

Thus, the invention also relates to peptide, protein, nucleic acids, vectors or host cells according to the invention for us in the preservation of the integrity of the endothelial cell junctions, in the protection of the occurrence of vascular alteration, thrombosis and inflammation during a bacterial infection.

In a particular embodiment, the invention also relates to peptide, protein, nucleic acids, vectors or host cells according to the invention for use in the protection of Primary Human Dermal Microvascular Endothelial Cells (HDMECs) or Primary Human Brain Microvascular Endothelial Cells (HBMECs).

Particularly, the invention relates to peptide, protein, nucleic acids, vectors or host cells according to the invention for use in the treatment of a sepsis, particularly due to (or induce by) a bacterial infection.

As used herein the term “Sepsis” means morbid condition induced by the mix of microbial virulence factors (such as toxins) and pro-inflammatory mediators, the introduction or accumulation of which is caused by infection or trauma, and includes the early stage of sepsis, severe sepsis and the acute phase of septic shock.

The term “early stage sepsis” refers to the stage of the disease with the onset of clinical symptoms of severe infectious disease that typically include chills, profuse sweat, irregularly remittent fever, prostration and the like.

The term “severe sepsis” refers to the stage of the disease with the clinical symptoms of early stage sepsis in addition to persistent fever, lymphopenia, disseminated intravascular coagulation, respiratory distress syndrome, multiple organ failure and hypotension leading to shock.

The term “acute phase of septic shock” refers to peripheral circulatory collapse, resulting in hemodynamic, metabolic and visceral disorders that almost invariably lead to death. Particularly, the peptides used to treat a sepsis can be the peptides as set forth in SEQ ID NO: 1 to 30 and 37 to 64 and particularly 1 and 8 to 30 and 37 to 64.

According to the present invention the bacterial infection is a Gram-negative bacterial infection or a Gram-positive bacterial infection.

The term “Gram-negative” bacterial infection refers to a local or systemic infection with Gram-negative bacteria. The proteobacteria are a major group of Gram-negative bacteria, including Escherichia coli (E. coli), Salmonella, Shigella, and other Enterobacteriaceae, Pseudomonas, Moraxella, Helicobacter, Stenotrophomonas, Bdellovibrio, acetic acid bacteria, Legionella etc. Other notable groups of gram-negative bacteria include the cyanobacteria, spirochaetes, green sulfur, and green non-sulfur bacteria. Medically relevant Gram-negative cocci include the four types that cause a sexually transmitted disease (Neisseria gonorrhoeae), a meningitis (Neisseria meningitidis), and respiratory symptoms (Moraxella catarrhalis, Haemophilus influenzae). Medically relevant Gram-negative bacilli include a multitude of species. Some of them cause primarily respiratory problems (Klebsiella pneumoniae, Legionella pneumophila, Pseudomonas aeruginosa), primarily urinary problems (Escherichia coli, Proteus mirabilis, Enterobacter cloacae, Serratia marcescens), and primarily gastrointestinal problems (Helicobacter pylori, Salmonella Enteritidis, Salmonella Typhimurium). Gram-negative bacteria associated with hospital-acquired infection include Acinetobacter baumannii, which cause bacteraemia, secondary meningitis, and ventilator-associated pneumonia in hospital intensive-care units.

In another particular embodiment of the invention, the Gram-negative bacteria according to the invention are selected from the group consisting of Escherichia coli, Pseudomonas spp, Salmonella spp, Klebsiella spp, Acinetobacter spp, E. corrodens, and Haemophilus influenza.

In a more particular embodiment of the invention, the Gram-negative bacteria according to the invention is Pseudomonas spp, Acinetobacter spp and Klebsiella spp.

The term “Pseudomonas bacteria” has its general meaning in the art and refers to bacteria that occur normally or pathogenically in lung of humans and other animals. The term “Pseudomonas bacteria” refers to but it is not limited to Gram-negative bacteria Pseudomonas, e.g.; a bacterium of the Pseudomonas aeruginosa group such as P. aeruginosa group.

In particular, the Pseudomonas according to the invention is Pseudomonas aeruginosa.

Pseudomonas aeruginosa is a common Gram-negative bacterium that can cause disease in animals, including humans. It is citrate, catalase, and oxidase positive. It is found in soil, water, skin flora, and most man-made environments throughout the world. It thrives not only in normal atmospheres, but also in hypoxic atmospheres, and has, thus, colonized many natural and artificial environments. It uses a wide range of organic material for food; in animals, its versatility enables the organism to infect damaged tissues or those with reduced immunity. The symptoms of such infections are generalized inflammation and sepsis. If such colonization occurs in critical body organs, such as the lungs, the urinary tract, and kidneys, the results can be fatal (Balcht, et al., Informa Health Care, 1994). Because it thrives on moist surfaces, this bacterium is also found on and in medical equipment, including catheters, causing cross-infections in hospitals and clinics.

The term “Klebsiella bacteria” has its general meaning in the art and refers to bacteria that occur normally or pathogenically in lung of humans and other animals. The term “Klebsiella bacteria” refers to but it is not limited to Gram-negative bacteria Klebsiella, e.g.; a bacterium of the Klebsiella pneumoniae group such as K. pneumoniae group, Klebsiella granulomatis, Klebsiella oxytoca, Klebsiella michiganensis, Klebsiella pneumoniae (species-type), Klebsiella pneumoniae subsp. ozaenae, Klebsiella pneumoniae subsp. pneumoniae, Klebsiella pneumoniae subsp. rhinoscleromatis, Klebsiella quasipneumoniae, Klebsiella quasipneumoniae subsp. quasipneumoniae, Klebsiella quasipneumoniae subsp. similipneumoniae, Klebsiella variicola.

In particular, the Klebsiella according to the invention is Klebsiella pneumoniae.

The term “Acinetobacter bacteria” has its general meaning in the art and refers to bacteria that occur normally or pathogenically in lung of humans and other animals. The term “Acinetobacter bacteria” refers to but it is not limited to gram-negative bacteria Acinetobacter, e.g.; a bacterium of the Acinetobacter baumannii group such as Acinetobacter baumannii, Acinetobacter calcoaceticus, Acinetobacter genomospecies 3 and Acinetobacter genomospecies 13 (Ingela Tjernberg et Jan Ursing) grouped together in a group called the “Acinetobacter calcoaceticus-baumannii complex”.

In particular, the Acinetobacter according to the invention is Acinetobacter baumannii.

The term “gram-positive” bacterial infection refers to a local or systemic infection with gram-positive bacteria. Gram-positive bacteria are bacteria that give a positive result in the Gram stain test, which is traditionally used to quickly classify bacteria into two broad categories according to their cell wall. Gram-positive bacteria take up the crystal violet stain used in the test, and then appear to be purple-coloured when seen through a microscope. This is because the thick peptidoglycan layer in the bacterial cell wall retains the stain after it is washed away from the rest of the sample, in the decolorization stage of the test.

In the classical sense, six Gram-positive genera are typically pathogenic in humans. Two of these, Streptococcus and Staphylococcus are cocci (sphere-shaped). The remaining organisms are bacilli (rod-shaped) and can be subdivided based on their ability to form spores. The non-spore formers are Corynebacterium and Listeria (a coccobacillus), whereas Bacillus and Clostridium produce spores (Gladwin, et al (2007). Miami, Florida: MedMaster. pp. 4-5. ISBN 978-0-940780-81-1).

In particular embodiment of the invention, the Gram-positive bacteria according to the invention are selected from the group consisting of Staphylococcus, Streptococcus, Clostridium, Listeria, Bacillus and Corynebacterium.

In another particular embodiment of the invention, the Gram-positive bacteria according to the invention is Staphylococcus selected from the group consisting of S. aureus group (S. argenteus, S. aureus, S. schweitzeri, S. simiae), S. auricularis group (S. auricularis), S. epidermidis group (S. capitis, S. caprae, S. epidermidis, S. saccharolyticus), S. haemolyticus group (S. borealis, S. devriesei, S. haemolyticus, S. hominis); S. hyicus-intermedius group (S. agnetis, S. chromogenes, S. cornubiensis, S. felis, S. delphini, S. hyicus, S. intermedius, S. lutrae, S. microti, S. muscae, S. pseudintermedius, S. rostri, S. schleiferi), S. lugdunensis group (S. lugdunensis); S. sciuri group (S. fleurettii, S. lentus, S. sciuri, S. stepanovicii, S. vitulinus), S. simulans group (S. simulans) and S. warneri group (S. pasteuri, S. warneri)

In more particular embodiment, the Gram-positive bacteria according to the invention is S. aureus group.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

The invention also relates to a method for treating an infectious disease in a subject in need thereof comprising administering to a subject in need thereof a therapeutically effective amount of a peptide, a protein, nucleic acids, vectors or host cells according to the invention.

### Kit of Part

A seventh aspect of the present invention relates to i) a peptide, a protein, nucleic acids, vectors or host cells according to the invention, and ii) at least one anti-pathogen infection agent, as a combined preparation for simultaneous, separate or sequential use in the treatment of an infectious disease in a subject in need thereof.

Particularly, the anti-pathogen infection agent can be an anti-viral infection agent, an anti-bacterial infection agent, an anti-protozoan infection agent, an anti-prion infection agent, a, anti-viroid infection agent, or an anti-fungus infection agent.

Thus, the invention also relates to i) a peptide, a protein, nucleic acids, vectors or host cells according to the invention, and ii) at least one anti-bacterial infection agent, as a combined preparation for simultaneous, separate or sequential use in the treatment of a bacterial infection in a subject in need thereof.

According to the anti-bacterial infection agent can be an antibiotic.

As used herein, the terms “antibiotic” and “antimicrobial compound” are used interchangeably and refer to a compound which decreases the viability of a microorganism, or which inhibits the growth or reproduction of a microorganism. The term “antibiotic agent” has its general meaning in the art and refers to antibacterial agent, such as described in US2013/0029981.

Suitable main class of antibiotic agents include, without limitation:

1. β-lactam antibiotic (beta-lactam antibiotic) are the antibiotic agents that contain a beta-lactam ring in their molecular structure and containing a beta-lactam functionality. These β-lactam antibiotics includes penicillin and derivatives (penams), cephalosporins (cephems), monobactams, carbapenems and carbacephems. Most β-lactam antibiotics work by inhibiting cell wall biosynthesis in the bacterial organism and are the most widely used group of antibiotics (in 2003 more than half of all commercially available antibiotics in use were β-lactam compounds).

By “cephalosporins” (cephems) is meant herein a subgroup of β-lactam antibiotics originally derived from the fungus Acremonium. Together with cephamycins, they constitute a subgroup of β-lactam antibiotics called cephems. Cephalosporins include ceftazidime.

By “monobactam” is meant herein a subgroup of β-lactam antibiotics, which are monocyclic and wherein the β-lactam ring is not fused to another ring. Monobactam include aztreonam.

By “carbapenems” is meant herein a subgroup of β-lactam antibiotics, which have a bactericide effect by binding to penicillin-binding proteins (CBPs) thus inhibiting bacterial cell wall synthesis This class of antibiotics is usually reserved for known or suspected multidrug-resistant (MDR) bacterial infections. Carbapenem include imipenem.

By “penicillin” and “penicillin derivatives” (penams) is meant herein a subgroup of β-lactam antibiotics, derived originally from common moulds known as Penicillium moulds; which includes penicillin G (intravenous use), penicillin V (use by mouth), procaine penicillin, and benzathine penicillin (intramuscular use). Penicillin antibiotics were among the first medications to be effective against many bacterial infections caused by staphylococci and streptococci. They are still widely used today, though many types of bacteria have developed resistance following extensive use. There are several enhanced penicillin families which are effective against additional bacteria; these include the antistaphylococcal penicillins, aminopenicillins and the antipseudomonal penicillins. They are derived from Penicillium fungi.

Example of Natural penicillin: Penicillin G, Penicillin K, Penicillin N, Penicillin O, Penicillin V.

Example of β-lactamase-resistant penicillin derivatives: Methicillin, Nafcillin, Oxacillin, Cloxacillin, Dicloxacillin, Flucloxacillin.

Example of Aminopenicillins: Ampicillin, Amoxicillin, Pivampicillin, Hetacillin, Bacampicillin, Metampicillin, Talampicillin, Epicillin.

Example of Carboxypenicillins: Carbenicillin, Ticarcillin, Temocillin.

Example of Ureidopenicillins: Mezlocillin, Piperacillin, Azlocillin.

Example of β-lactamase inhibitors penicillin derivatives: Clavulanic acid, Sulbactam, Tazobactam.

2. Aminoglycoside are the antibiotic agents directed to Gram negative bacteria that inhibit protein synthesis (targeting the small ribosome sub-unit of 30) and contain as a portion of the molecule an amino-modified glycoside (Mingeot-Leclercq M P, et al (1999). Antimicrob. Agents Chemother. 43 (4): 727-37). The term “Aminoglycoside” can also refer more generally to any organic molecule that contains amino sugar substructures. Aminoglycoside antibiotics display bactericidal activity against Gram-negative aerobes and some anaerobic bacilli where resistance has not yet arisen but generally not against Gram-positive and anaerobic Gram-negative bacteria.

Streptomycin is the first-in-class aminoglycoside antibiotic. It is derived from Streptomyces griseus and is the earliest modern agent used against tuberculosis. Streptomycin lacks the common 2-deoxystreptamine moiety present in most other members of this class. Other examples of aminoglycosides include the deoxystreptamine-containing agents, kanamycin, tobramycin, gentamicin, and neomycin.

3. Antibiotic agents which inhibit acid nucleic synthesis
Antibiotic agent which block DNA gyrase (topoisomerase specific to bacteria): aminocoumarines, and quinolones.
Antibiotic agents which block the bacterial RNA polymerase: rifampicine.

4. Antibiotics which inhibit protein synthesis (other than Aminoglycoside)
Antibiotic agents which block the formation of the peptide bond: amphenicols (examples: chloramphenicol, thiamphenicol azidamfenicol and florfenicol)
Antibiotic agents which block elongation of the polypeptide chain: Tetracyclins (examples: tetracycline, doxycycline, aureomycine, eravacycline, sarecycline, omadacycline) macrolides (examples: erythromycin, azithromycin) and ketolides (examples: telithromycin, cethromycin and solithromycin).

5. Antibiotics which inhibit folate metabolism

Sulfonamides also called sulphonamides, sulfa drugs or sulpha drugs (examples: Sulfamethoxazole) and sulfanilamides.

6. Antibiotics of the glycopeptide family like the vancomycin.

7. New classes of antibiotics compounds

Four new classes of antibiotics have been brought into clinical use in the late 2000s and early 2010s: cyclic lipopeptides (such as daptomycin), glycylcyclines (such as tigecycline), oxazolidinones (such as linezolid), and lipiarmycins (such as fidaxomicin).

In a particular embodiment, the antibiotic of the invention is the cefotaxime and an aminoglycoside (amikacin).

According to the invention and in the case of a sepsis, an anti-sepsis agent can be used.

Thus, in this case, the invention relates to i) a peptide, a protein, nucleic acids, vectors or host cells according to the invention, and ii) at least one anti-sepsis agent, as a combined preparation for simultaneous, separate or sequential use in the treatment of a sepsis in a subject in need thereof.

As use herein, the term “anti-sepsis agent” denotes compounds able to fight against the sepsis and are for example antibiotics, vasopressors (like norepinephrine or dopamine), hydrocortisone and fludrocortisone.

As used herein, the term “simultaneous use” denotes the use of a peptide, a protein, nucleic acids, vectors or host cells according to the invention and at least one anti-bacterial infection agent occurring at the same time.

As used herein, the term “separate use” denotes the use of a peptide, a protein, nucleic acids, vectors or host cells according to the invention and at least one anti-bacterial infection agent not occurring at the same time.

As used herein, the term “sequential use” denotes the use of a peptide, a protein, nucleic acids, vectors or host cells according to the invention and at least one anti-bacterial infection agent occurring by following an order.

### Therapeutic Composition

Another object of the invention relates to a therapeutic composition comprising an antibody or a peptide, a protein, nucleic acids, vectors or host cells according to the invention for use in the treatment of an infectious disease in a subject in need thereof.

In a particular embodiment, the invention relates to a therapeutic composition comprising a peptide, a protein, nucleic acids, vectors or host cells according to the invention for use in the treatment of a sepsis in a subject in need thereof.

Any therapeutic agent of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intrathecal, intramuscular or subcutaneous administration or lung nebulization and the like.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

In addition, other pharmaceutically acceptable forms include, e.g. tablets or other solids for oral administration; time release capsules; and any other form currently can be used.

Pharmaceutical compositions of the present invention may comprise a further therapeutic active agent. The present invention also relates to a kit comprising an agonist, antagonist or inhibitor of the expression according to the invention and a further therapeutic active agent.

For example, anti-bacterial infection agent like antibiotics or anti-sepsis (as described above) agents may be added to the pharmaceutical composition as described below.

The invention will be further illustrated by the following figures and examples. However, these examples and figures should not be interpreted in any way as limiting the scope of the present invention.

### FIGURES

FIG. 1. Exogenous ANGPTL4 exerts protective effects on dermal microvascular endothelial cells infected by N. meningitidis. A. HDMECs were infected with Nm 2C4.3 for 30 min and treated with 1 μg/mL of recombinant human ANGPTL4 for 2 h 30. Cells were fixed and stained for cellular actin, VE-cadherin and DAPI and analyzed by fluorescent microscopy. Quantifications of VE-cadherin enrichment at intercellular junctions and of intercellular spaces area were performed on 30 fields with Image J software. Kruskal-Wallis test ** P<0.001; P<0.0001. B. HDMEC were either uninfected or infected with N. meningitidis 2C4.3 for 30 min and treated with 1 μg mL₋₁ of recombinant human ANGPTL4, its N-terminal portion (nANGPTL4) or its C-terminal portion (cANGPTL4) for 2 h 30. Caspase-3/7 reagent was added to detect apoptosis in real time over 9 h using the IncuCyte® Live-Cell Analysis System

FIG. 2: Vascular protection is conferred by the N-terminal part of ANGPTL4. A, Schematic representation of ANGPTL4 structure. ANGPTL4 contains a N-terminal portion (nANGPTL4, aa 26-164) linked to a C-terminal portion (cANGPTL4, aa 164-406) by a cleavable linker. nANGPTL4 contains a highly conserved lipoprotein lipase (LPL) binding domain (aa 44-55), two cysteines on position 76 and 80 involved in protein oligomerization and a coiled-coil domain (aa 100-143). The C-terminal portion encodes a fibrinogen-like domain. B, HDMECs were infected with Nm 2C4.3 for 30 min and treated with 1 μg/mL of recombinant human ANGPTL4, nANGPTL4 or cANGPTL4 for 2 h 30. Cells were fixed and stained for cellular actin, VE-cadherin and DAPI and analyzed by fluorescent microscopy. B. Quantifications of VE-cadherin enrichment at intercellular junctions and of intercellular spaces area were performed on 30 fields with Image J software. Kruskal-Wallis test ** P<0.001, *** P<0.0001 of four independent experiments performed in triplicate.

FIG. 3: Vascular protection is conferred by a novel binding motif (aa 66-80) present in the N-terminal part of ANGPTL4. A, the indicated peptides were derived from nANGPTL4. B, HDMECs were infected with Nm 2C4.3 for 30 min and treated with 1 μg/mL of each peptides. Quantifications of VE-cadherin enrichment at intercellular junctions was performed on 30 fields with Image J software.

FIG. 4: Vascular protection is conferred by interaction of nANGPTL4 with Syndecan-4. A, HDMECs were infected with Nm 2C4.3 for 30 min and treated with 1 μg/mL of recombinant human ANGPTL4, nANGPTL4 or cANGPTL4 in the absence or in the presence of anti-SDC4 antibody for 2 h 30. Cells were fixed and stained for cellular actin, VE-cadherin and DAPI and analysed by fluorescent microscopy. Quantifications of VE-cadherin enrichment at intercellular junctions and of intercellular spaces area were performed on 30 fields with Image J software. Kruskal-Wallis test ** P<0.001, **** P<0.00001, ns1 P=0.2172; ns2 P>0.999 of four independent experiments performed in triplicate. B, HDMECs were transfected with small interfering RNA (siRNA) against SDC4 or non-targeting siRNA controls for 48 h, infected with Nm 2C4.3 for 30 min and treated with 1 μg/mL of recombinant human ANGPTL4, for 2 h 30. Quantifications of VE-cadherin enrichment at intercellular junctions and of intercellular spaces area were performed on 30 fields with Image J software. Kruskal-Wallis test ** P<0.001, *** P<0.0001, of two independent experiments performed in triplicate.

FIG. 5: Antibody neutralisation of SDC4 in brain endothelial cells induces the loss of endothelial integrity in response to meningococcal infection. HCMECs/D3 were infected with Nm 2C4.3 for 30 min and treated with 10 μg/mL of anti-ANGPTL4 antibody for 2 h 30 in the absence or in the presence of anti-SDC4 antibody, or anti-ANGPTL4 antibody as a positive control. A, Quantifications of VE-cadherin enrichment at intercellular junctions and of intercellular spaces area were performed on 30 fields with Image J software. Kruskal-Wallis test ** P<0.001, *** P<0.0001. B, human primary human brain microvascular endothelial cells (HBMECs) were grown on cell filters. Cells were infected with Nm 2C4.3 for 30 min and treated with 10 μg/mL of anti-ANGPTL4 antibody for 2 h 30 in the absence or in the presence of anti-SDC4 antibody, or anti-ANGPTL4 antibody. The transendothelial electrical resistance (TEER) was measured. Shown are the result of one representative experiment of two independent experiments performed in triplicate. Kruskal-Wallis test P<0.001.

FIG. 6: ANGPTL4 protects dermal microvascular endothelial cells from infection by S. pneumoniae. HDMECs were infected with S. pneumoniae TIGR4 for 30 min and treated with 1 μg/mL of recombinant human ANGPTL4 for 2 h. Cells were fixed and stained for cellular actin, VE-cadherin and DAPI and analyzed by fluorescent microscopy. Quantifications of VE-cadherin enrichment at intercellular junctions was performed on 30 fields with Image J software. Kruskal-Wallis test *** P<0.0001.

FIG. 7: ANGPTL4 reduces signs of thrombosis, vascular injury and inflammation. A-D, SCID mice grafted with human skins were infected intravenously with Nm 2C4.3 wild-type strain (5×10⁶ bacteria) or left uninfected. A period of 30 min or 2 h after bacterial challenge, mice received 1 μg of recombinant human ANGPTL4, nANGPTL4 or cANGPTL4 intravenously or the vehicle as a control. Mice were killed at 4 h post-challenge. This experiment was performed twice with n=3 or 4 mice per group, using skin from two different donors. A, A schematic representation of the protocols used in vivo. B, Control bacteraemia 4 h post-infection (mean±s.e.m.). C, Bacterial colonization within the skin grafts as assessed by immunofluorescence analysis was quantified using ImageJ software as the ratio between bacterial surface colonization and the lumen surface of the vessels. A total of 30-50 vessels per mouse were analysed. Error bars show mean±s.e.m. D, Thrombus formation within the skin grafts was assessed by immunofluorescence analysis and quantified using ImageJ as the ratio between thrombus surfaces and the lumen surface of the vessels. A total of 30-50 vessels per mouse were analysed. Error bars show mean±s.e.m.; *** P<0.0001; ns P=0.0541 two-tailed Student's t-test.

FIG. 8: ANGPTL4 improves the outcome of meningococcal infection alone or in combination with antibiotics. A-D, SCID mice grafted with human skin were infected intravenously with Nm 2C4.3 wild-type strain (5×10⁶ bacteria); 2 h after bacterial challenge, mice received recombinant human ANGPTL4 (1 μg, intravenously), cefotaxime (200 mg kg-1, intraperitoneally) alone or in combination with ANGPTL4 or vehicle as a control. 18 h following bacterial challenge, ANGPTL4 (1 μg) and cefotaxime (200 mg kg-1) were re-administered. This experiment was performed twice with n=3 or 4 mice per group, using skin from two different donors. A, A schematic representation of the protocol used in vivo. B, Bacteraemia 4, 18, 48 and 72 h post-infection (mean±s.e.m.). C, Survival curve: * P=0.0275; two-sided log-rank Mantel-Cox survival analysis. D, Thrombosis within the skin grafts at 4 h post-infection was assessed by immunofluorescence analysis and quantified as described in FIG. 8 (error bars show mean±s.e.m.; 40 vessels per mouse were analysed; n=4 mice per group; *** P<0.001; two-tailed Student's t-test.

FIG. 9: ANGPTL4 protects against LPS-induced endotoxemia by preventing vascular dysfunction and organ failure. A-B, Balb/c mice received LPS (5 mg kg-1, i.p.); 30 min after LPS challenge, mice received recombinant human ANGPTL4 (2 μg, intravenously), or vehicle as a control. A, A schematic representation of the protocol used. B, Survival curve (n=16 mice per group): **** P<0.0001; two-sided log-rank Mantel-Cox survival analysis. C,D, Balb/c mice received LPS (5 mg kg-1, i.p.); 30 min after LPS challenge, mice received nANGPTL4 (2 μg, intravenously), or vehicle as a control. C, A schematic representation of the protocol used. D, Survival curve (n=6 mice per group): ** P=0.0033; two-sided log-rank Mantel-Cox survival analysis. E, F, Balb/c mice received LPS (5 mg kg-1, i.p.); 30 min after LPS challenge, mice received Peptide 1 (2 μg, intravenously), or vehicle as a control. E, A schematic representation of the protocol used. F, Survival curve (n=6 mice per group): * P=0.0163; two-sided log-rank Mantel-Cox survival analysis. G, Balb/c mice received LPS (5 mg kg-1. i.p.): 30 min after LPS challenge, mice received recombinant human peptide P9 (2 μg. intravenously). or vehicle as a control. Survival curves (n=6 mice per group). *** P<0.001: two-sided log-rank Mantel-Cox survival analysis. H. Balb/c mice received LPS (4 mg kg-1. i.p.) together with anti-ANGPTL4 blocking antibody (10 μg kg-1. i.v) or vehicle as a control. 18 h after LPS challenge. mice were perfused with Evans blue for 1 h. were killed and brain were extracted. Quantification analysis of Evans blue extravasation (n=4 mice per group). Bars and error bars show the mean±s.e.m., respectively, from two independent experiments: *** P<0.001: NS, not significant (P>0.05) one-way ANOVA Tukey's multiple comparison test.

TABLE 1
Effect of nANGPTL4-derived peptides on endothelial cell junction stabilization

| | Lenght | Positions | | Junctional |
|---|---|---|---|---|
| Peptide | (aa) | (aa) | Sequence | stabilization |
| P1 | 46 | 38-83 | WDEMNVLAHGLLQLGQGLREHAERTR | + |
| | | | SQLSALERRLSACGSACQGT (SEQ ID | |
| | | | NO: 20) | |
| P2 | 50 | 84-148 | PEVLHSLQTQLKAQNSRIQQLFHKVAQ | − |
| | | | QQRHLEKQHLRIQHLQSQFGLLD (SEQ | |
| | | | ID NO: 31) | |
| P1 | 46 | 38-83 | WDEMNVLAHGLLQLGQGLREHAERTR | + |
| C76A/C80A | | | SQLSALERRLSAAGSAAQGT (SEQ ID | |
| | | | NO: 21) | |
| P3 | 18 | 38-55 | WDEMNVLAHGLLQLGQGL (SEQ ID | − |
| | | | NO: 32) | |
| P4 | 28 | 56-83 | REHAERTRSQLSALERRLSACGSACQG | + |
| | | | T (SEQ ID NO: 16) | |
| P5 | 19 | 56-73 | REHAERTRSQLSALERRL (SEQ ID NO: | − |
| | | | 33) | |
| P6 | 16 | 65-80 | QLSALERRLSACGSAC (SEQ ID NO: | + |
| | | | 12) | |
| P7 | 15 | 61-75 | RTRSQLSALERRLSA (SEQ ID NO: 34) | − |
| P8 | 10 | 66-75 | LSALERRLSA (SEQ ID NO: 35) | − |
| P9 | 15 | 66-80 | LSALERRLSACGSAC (SEQ ID NO: 8) | + |
| P10 | 12 | 67-78 | SALERRLSACGS (SEQ ID NO: 36) | − |
| P12 | 13 | 66-78 | LSALERRLSACGS (SEQ ID NO: 55) | + |
| P13 | 14 | 67-80 | SALERRLSACGSAC (SEQ ID NO: 58) | + |

### EXAMPLE

### Material & Methods

### Antibodies, Recombinant Proteins and Reagents

Anti-Collagen IV (ab6311) mouse monoclonal antibody and anti-CD41 (ab33611) rat monoclonal antibody were purchased from Abcam. Anti-VE-Cadherin F-8 Alexa Fluor® 647 mouse monoclonal antibody (sc-9989) was purchased from Santa Cruz. Anti-Syndecan 4 rabbit polyclonal antibody was purchased from Invitrogen (36-3100). Anti-Angptl4 rabbit polyclonal antibody was purchased from Thermo scientific (40-9800). Polyclonal antiserum raised against meningococcal 2C4.3 strain was described previously [22]. Secondary antibodies used for immunofluorescence labelling and immunoblotting were from Jackson ImmunoResearch Laboratories and Thermoscientific Lab.

Recombinant human ANGPTL4 (rhANGPTL4, 44487-AN), C-terminal fragment (cANGPTL4,3485-AN), N-terminal fragment (nANGPTL4, 8249-AN), rhANGPTL3 (3829-AN) or rhANGPTL8 (9983-AN) were purchased from R&D Systems. LPS from Escherichia coli (0111: B4) was purchased from Sigma-Aldrich. The peptides were all synthesized by Covalab (Lyon, France) with a purity >98%.

### Bacterial Strain and Infection

Neisseria meningitidis (Nm) 2C4.3 strain, (formerly clone 12) that is a piliated capsulated Opa− Opc− variant of the serogroup C meningococcal clinical isolate 8013 was described before [23]. Bacterial strains were stored frozen at −80° C. and routinely grown at 37° C. under 5% CO2 on GC agar plates (Difco) containing Kellogg's supplements.

The day of infection, a suspension of the bacteria from an overnight culture on GCB agar plate was adjusted to OD600=0.05 and incubated for 2 h at 37° C. in a pre-warmed cell culture medium. Cells were infected with bacteria at a multiplicity of infection (MOI) of 100 bacteria per cell (OD=0.1) for 30 minutes, washed twice to remove non-adherent bacteria and infection was allowed to proceed for various periods of time. Cells were then washed and fixed in 4% paraformaldehyde for immunofluorescence analysis.

### Cell Lines

Human Cerebral Microvascular Endothelial Cells (HCMECs/D3) are fully differentiated brain endothelial cell line derived from human brain capillaries, engineered in our laboratory, and which recapitulate the major phenotypic features of the blood-brain barrier [13, 24]. HCMEC/D3 were grown onto cultrex rat collagen type I-coated dishes (R&D) in Endothelial Cell Basal Medium-2 (Lonza) supplemented with 5% of FCS, 1.4 μM hydrocortisone (Lonza), 5 μg/mL ascorbic acid (Lonza), 1 ng/mL b-FGF (Lonza), at 37° C. in 5% CO2.

Primary Human Brain Microvascular Endothelial Cells (HBMECs) isolated from cerebral biopsy within healthy areas of pediatric brain tumor, and their specific endothelial cell growth medium, and grown at 37° C. in 5% CO2.

Primary Human Dermal Microvascular Endothelial Cells (HDMECs) isolated from the dermis of juvenile foreskin and adult skin (different locations) and their specific endothelial cell growth medium were purchased from PromoCell, and grown at 37° C. in 5% CO2. When indicated, HDMECs were split on channel slides (μ-slide 0.4 I Luer from ibidi), grown for 2 d to reach confluency and then subjected to a laminar flow shear stress (10 dyn cm-2) for 4 d (ibidi Pump System, ibidi)

### Confocal Immunofluorescence Microscopy

Cells were grown to confluence on Thermanox coverslips (Thermo Fischer Scientific). After infection, cells were fixed in 4% paraformaldehyde for 10 min, washed three times with PBS and permeabilized with 0.2% Triton X-100 in PBS for 10 min. Cells were blocked for 30 min with 3% BSA in PBS and were incubated for 2 h with the primary antibodies. After three washes with PBS, cells were incubated for 1 h with CY3-conjugated anti-mouse IgG and CY2-conjugated anti-rabbit IgG (Jackson Immunochemicals). Image acquisitions were performed on a confocal microscopy (spinning disk Leica DMI6000, ×20). Images analyses were carried out with the ImageJ Software (NIH). Each experiment was repeated at least three times.

Infection of SCID Mice Grafted with Human Skin.

Six-week-old CB17/Icr-Prkdcscid female mice were obtained from Janvier Labs. Human skin tissues were obtained from adult patients undergoing plastic surgery in the Service de Chirurgie Plastique et Reconstructive of the Groupe Hospitalier Saint-Joseph. In accordance with French legislation, patients were informed and did not refuse to participate in the study. Experimental procedures were performed as previously described [25] in accordance with the guidelines of the Institut National de la Santé et de la Recherche Médicale and conformed to the European ethical legislation (Directive 2010/63/EU). The experimental protocol was approved by the Animal Experimentation Ethics Committee of the Université Paris Descartes (consent forms CEEA34.O.J.L.039.12 and 2018012515596498). Briefly, mice were prepared for transplantation by shaving the hair on the back and abdominal areas after an intraperitoneal injection of ketamine (100 mg kg-1) and xylazine (10 mg kg-1). A skin flap was created and a full thickness human skin graft was placed onto the wound bed. The transplants were held in place with 6-0 non-absorbable monofilament suture materials, and the flap was then sutured above the transplant. Grafted mice were infected 4-6 weeks after human skin transplantation. N. meningitidis was grown overnight at 37° C. on GCB agar plates prepared without iron and supplemented with 15 μM deferoxamine (Desferal, Novartis). Bacterial colonies were harvested and cultured in RPMI media with 1% BSA medium and 0.06 μM deferoxamine with gentle agitation to reach the exponential phase of growth. Bacteria were then resuspended in physiological saline solution. Mice were infected intravenously with 200 μl (5×10⁶ bacteria) of this bacterial suspension. A 10 mg dose of human holotransferrin (R&D Systems) was administered intraperitoneally just before infection. Mice were killed at 4 h after infection. Human skin grafts were carefully collected using a sterile cutter. The tissue was washed in PBS and fixed overnight at 4° C. in 4% paraformaldehyde in phosphate buffer. After washing in phosphate buffer, the specimens were embedded in OCT medium and then frozen at −80° C. Sections of the dermis (7 μm thick) were immobilized on Superfrost Plus microscope slides and subjected to immunofluorescence analysis. Sections were incubated with the primary antibodies for 1 h in PBS/3% BSA then DAPI was added to Alexa-conjugated secondary antibodies for 1 h. After additional washing, coverslips were mounted in glycergel (Dako) and were further analysed using a slide scanner (Lamina, Perkin Elmer). Analysis of the images was performed using ImageJ software (NIH).

### LPS-Induced Sepsis in Mice

Female BALB/c mice, obtained from Janvier Labs, 9-10 weeks of age, were administered LPS at 5 mg/kg intraperitoneally and survival was monitored for 72 h (n=12 per group). BALB/c mice were randomly divided into 2 groups: LPS and LPS+Angptl4. Angptl4 (2 μg/mice) or vehicle (physiological saline solution) was administered intravenously 30 min after LPS challenge. In some experiments, blood serum, kidney and lung were collected at 18 h post-LPS challenge. Kidney and lungs were fixed in 4% PFA, embedded in paraffin, and 5-μm sections were cut from each block and stained with H&E. The stained sections were observed and acquired using a slide scanner (Lamina, Perkin Elmer). Analysis of the images was performed using ImageJ software (NIH).

### Statistical Analysis

Data were examined for significance using Prism Software (GraphPad Software). Statistical significance was assessed with the Student's t-test and one-way ANOVA. Quantitative analyses were conducted on 3 independent experiments using ImageJ software.

### Results

Endogenous ANGPTL4 Preserves Brain Microvascular Endothelial Cells from Infection by Neisseria meningitidis.

By performing a transcriptional analysis of human primary human dermal (HDMECs) or brain (HBMECs) microvessels, which were uninfected or infected in vitro with meningococci, we observed that mRNA level of ANGPTL4 was more abundant in HBMECs, as compared to HDMECs and was further increased in HBMECs upon meningococcal infection, while it remained unchanged in HDMECs (data not shown). These differences were confirmed at the protein level (data not shown).

We therefore analysed the effect of the endogenous production of ANGPTL4 on the integrity of brain endothelial cells, using hCMEC/D3 cells, a human endothelial cell line derived from cerebral microvessels, which preserved most of the structural and functional features of the primary brain endothelial cells [12, 13]. As for primary brain endothelial cells, HCMEC/D3 cells constitutively express ANGPTL4 and this production was further induced upon meningococcal infection (data not shown). While these cells resisted to infection by meningococci for 3 h, with a maintained junctional organization of VE-Cadherin, a major component of adherens junctions that control vascular permeability, the addition of anti-ANGPTL4 blocking antibody induced the loss of junctional distribution of VE-cadherin and the loss of monolayer integrity (data not shown). These results indicated that ANGPTL4 secreted by brain microvascular cells preserves endothelial integrity in response to N. meningitidis infection.

Exogenous ANGPTL4 Protects Dermal Microvascular Endothelial Cells from Meningococcal Infection

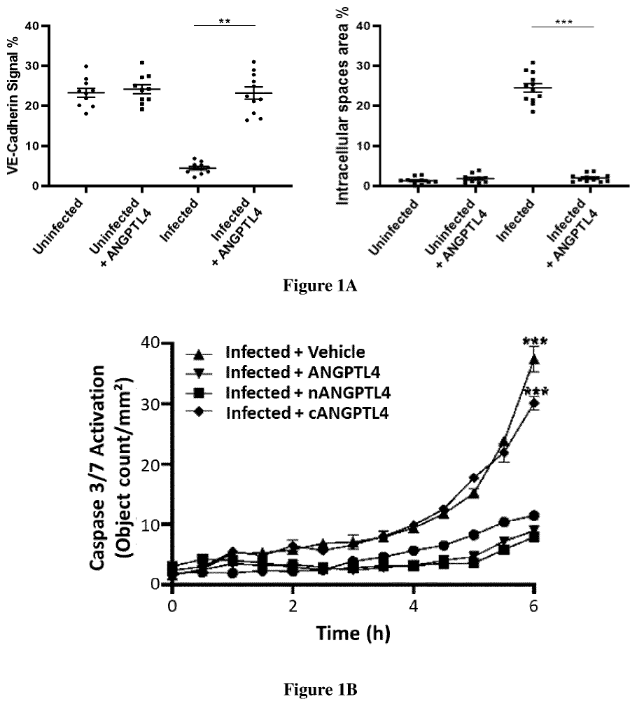

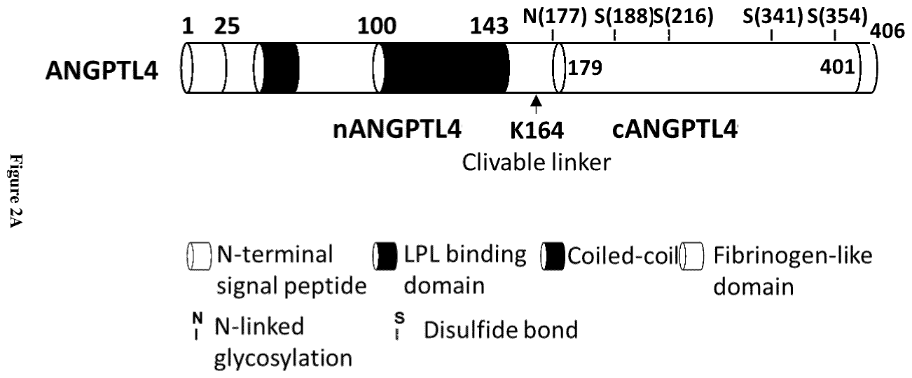

In contrast to brain microvascular endothelial cells that resist to meningococcal infection, the integrity of dermal microvascular endothelial cell (HDMECs) monolayer was massively impaired upon infection, as observed with the loss of adherens junctions and cell detachment (data not shown). Since HDMEC poorly produces ANGPTL4 as compared to HBMEC (data not shown), we analysed whether the addition of recombinant human ANGPTL4 might exert a protective effect on these cells. While treatment with ANGPTL4 had no significant effect on the organization of the adherens junctions formed by HDMECs, addition of ANGPTL4, 30 min after the initiation of infection, entirely preserved the integrity of this monolayer significantly reduced Src kinase and VE-cadherin phosphorylation (FIG. 1A), and inhibited cell apoptosis induced by meningococcal infection (FIG. 1B) . . . . Additionally, while infection was accompanied by the massive release of inflammatory cytokines (TNF-α, IL-6, IL-8), markers of vascular inflammation (E-selectin, ICAM-1, VCAM-1), and markers of endothelial cell alteration (Endoglin, Thrombomodulin), all these markers were reduced by 35 to 80% upon treatment with ANGPTL4 (data not shown). Although ANGPTL4 shares sequence homology, biochemical and functional properties with ANGPTL3 and ANGPTL8 [6], addition of rhANGPTL3 or rhANGPTL8 had no protective effect on infected HDMECs (data not shown), demonstrating that this effect was specific to ANGPTL4. Altogether these results establish the potent cytoprotective effect of ANGPTL4 on meningococcal-induced endothelial dysfunction.

### Vascular Protection is Conferred by a Novel Binding Motif in the N-Terminal Part of ANGPTL4.

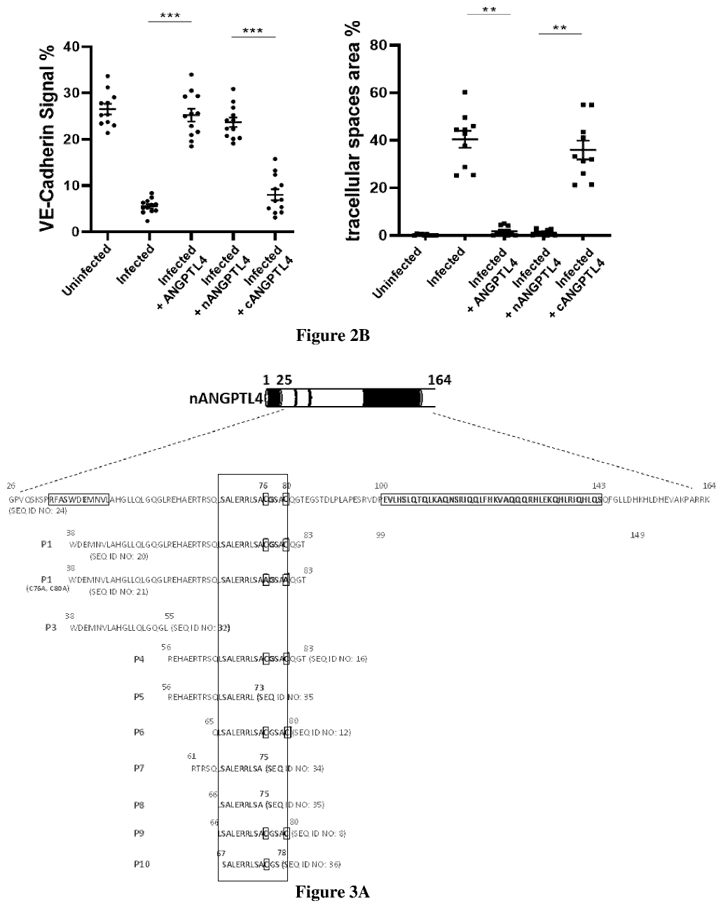

Endothelial cell-produced ANGPTL4 undergoes proteolytic processing by proprotein convertases at the linker region (Lysine 164) [14], releasing a N-terminal portion (nANGPTL4, aa 26-164) and a C-terminal portion (cANGPTL4, aa 164-406) (FIG. 2A) (the numbering of the amino acids is according to the entire sequence of ANGPTL4 (SEQ ID NO: 1)). nANGPTL4 contains a highly conserved lipoprotein lipase (LPL) binding domain (aa 44-55) that allow inhibition of LPL activity modulating lipid metabolism [15], two cysteines on position 76 and 80 involved in protein oligomerization through intermolecular disulfide bonds that are required for functional LPL inhibition, and a coiled-coil domain (aa 100-143) with no known function, while the monomeric C-terminal portion encodes a fibrinogen-like domain involved in protein interaction with various receptors (extracellular matrix proteins, integrins) and was shown to increase angiogenesis, vascular permeability and ROS (reactive oxygen species) production [16]. We then analysed the respective role of these domains in the protective effect of ANGPTL4 on infection-induced endothelial alterations. Addition of recombinant human nANGPTL4 was sufficient to maintain cell monolayer integrity upon infection, as observed with the full-length protein (FIG. 2B). Conversely, the addition of recombinant human cANGPTL4 had no significant protective effect on monolayer integrity, which remained altered upon infection (FIG. 2B).

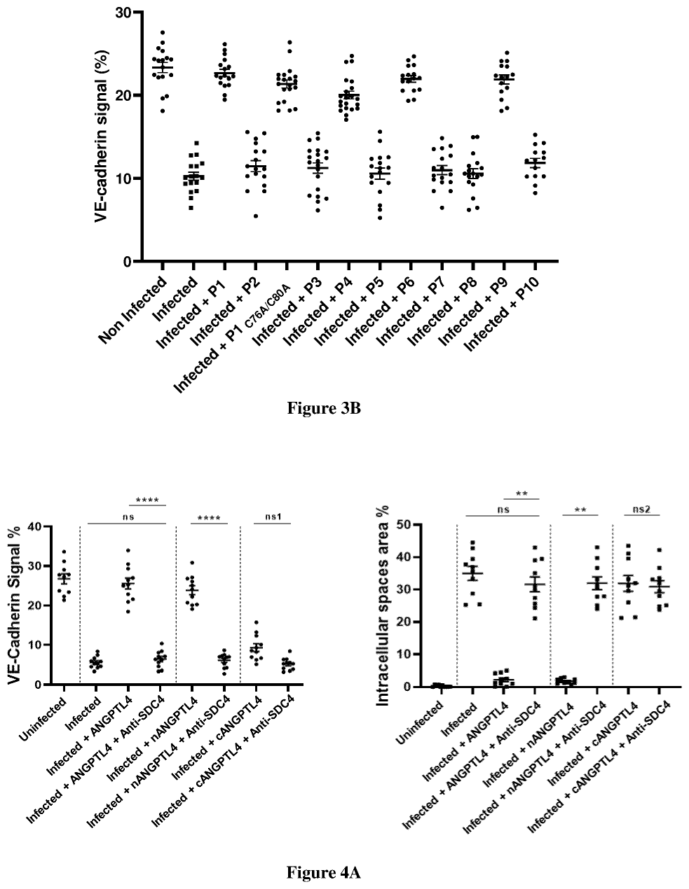

To identify the minimal interacting motif of nANGPTL4 required to promote vascular protective effects, we analysed the effects of derived peptides (FIG. 3A). At first, two peptides were tested: P1 (aa 38-83) containing the conserved LPL binding domain and the 2 cysteines 76 and 80, and P2 (aa 99-149) containing the coiled-coil domain. P1 was sufficient to prevent loss of adherens junctions and cell detachment induced by infection as efficiently as the full-length protein, while P2 had no significant effect (FIG. 3B). Interestingly, a P1 peptide containing mutations of the two cysteines in alanines (P1C76A, C80A) also efficiently provided cytoprotection to infection-induced damages, indicating that peptide oligomerization was not required to ensure this function (FIG. 3B). We then examined the effects of two P1-derived peptides: P3 (aa 38-55) had no protective effects indicating that the conserved LPL binding motif was not involved in this process, while P4 (aa 56-83) was as efficient as P1 in providing protection (FIG. 3B). Sequence plot analysis of P4 using PSIPRED predicted a potential membrane interaction domain encompassing aa 65-80. P6 (aa 65-80) and P9 (aa 66-80) encompassing this potential domain completely inhibited infection-induced damages, while P5 (aa 56-73), P7 (60-75) or two shorter peptides P8 (aa 66-75) and P10 (aa 67-78) had no effect (FIG. 3B), and the synthesis of peptide P11 (aa 69-80) failed, most likely due to hydrophobicity within the sequence causing inter- or intramolecular aggregation or secondary structure formation. We then shows that peptides P12 (aa 66-78) and P13 (aa 67-80) also inhibits infection-induced damages.

These results (summarized in table 1) provided evidence that exogenous ANGPTL4 can exert a major protective effect on the integrity of endothelial cell monolayers infected with N. meningitidis, and that this effect was conferred by a novel binding motif (aa 66-80) we identified in the N-terminal part of the protein.

Vascular Protection is Conferred by Interaction of nANGPTL4 with Syndecan-4.

As the N-terminal part of ANGPTL4 was previously shown to bind to heparan sulfate proteoglycans and, in particular, to Syndecan-4 (SDC4) [17], we investigated the potential role of SDC4 in the protective effects of ANGPTL4. Interestingly, SDC4 mRNA and protein were expressed in both brain (HBMECs, hCMEC/D3) and dermal (HDMECs) microvascular endothelial cells and increased upon infection by N. meningitis, with a much stronger induction in HDMECs (data not shown) associated with an increased interaction of SDC4 with ANGPTL4, as revealed by an enriched co-immunoprecipitation (data not shown).

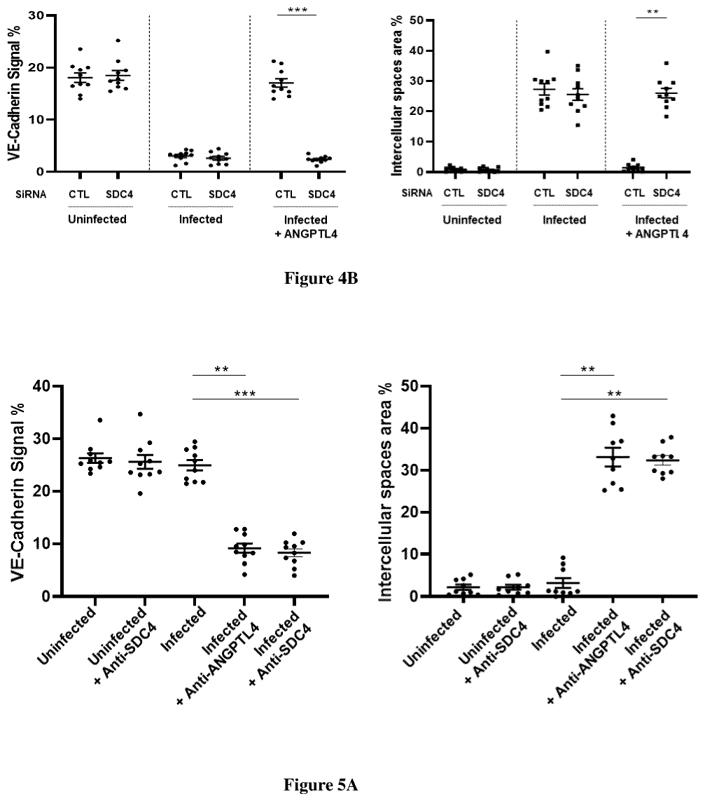

Co-treatment of HDMECs with ANGPTL4, nANGPTL4, in combination with an anti-SDC4 antibody, abolished the protective effect of both ANGPTL4 and nANGPTL4 on vascular alterations induced by infection, while anti-SDC4 antibody alone had no deleterious effects on uninfected cells (FIG. 4A). Anti-SDC4 antibody also abolished the protective effects of peptides P1 and P6 (data not shown), whereas anti-SDC1 antibody had no antagonistic effect on HDMECs treated with ANGPTL4, nANGPTL4 or derived peptides (data not shown), demonstrating that this effect was specific to SDC4. In addition, siRNA-mediated depletion of SDC4 in HDMECs prior to infection also abolished ANGPTL4 protective effects (FIG. 4B). Together, these results demonstrate that nANGPTL4 exerts a protective effect on the integrity of endothelial cell monolayers infected with N. meningitidis by interacting with SDC4 through its binding motif (aa 66-80).

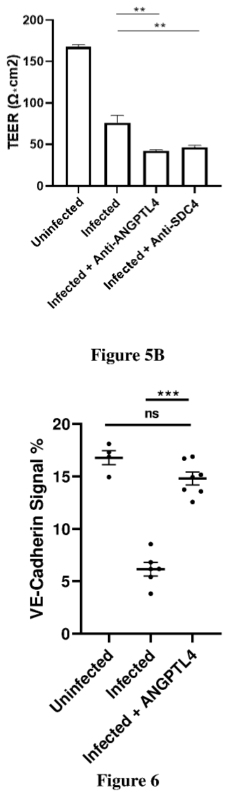

Similarly, addition of anti-SDC4 antibody to HCMEC/D3 cells induced the loss of junctional distribution of VE-cadherin and the loss of monolayer integrity in response to meningococcal infection, while no deleterious effects were observed on uninfected monolayers (FIG. 5A). These effects were further confirmed using primary brain endothelial cells. While infection by meningococci for 3 h only partially affected the strength and integrity of the barrier formed by HBMECs, as assessed by only 50% reduction in the transendothelial electrical resistance (TEER) and a maintained junctional organization of VE-Cadherin, addition of an anti-ANGPTL4 or anti-SDC4 blocking antibody 30 min after the initiation of infection similarly reduced TEER by 80% and induced disrupted junctions (FIG. 5B). This further indicates functional interaction of SDC4 and ANGPTL4 in brain endothelial cells conferring protection to bacterial infection.

Together, these data demonstrate the protective effect of ANGPTL4 on endothelial cell alterations promoted by meningococcal infection. To address whether ANGPTL-4 could also provide vascular protection from Gram-positive bacteria, we investigated the effect of ANGPTL-4 on HDMECs exposed to infection by the Gram-positive bacterium Streptococcus pneumoniae, one of the major causative agents of pneumonia, sepsis, meningitis and other morbidities. While S. pneumoniae infection induced the loss of endothelial cell junction organization, the addition of recombinant human ANGPTL4 preserved endothelial integrity (FIG. 6). These results indicate that ANGPTL4 is a potent vascular stabilization factor that may confer protection from both Gram-negative and Gram-positive bacterial infection.

### ANGPTL4 Prevents the Formation of Vascular Lesions In Vivo.

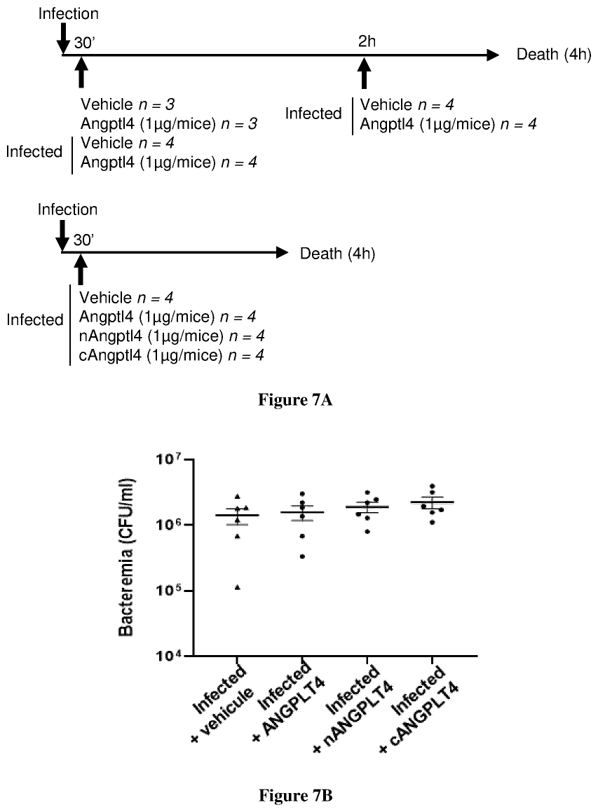

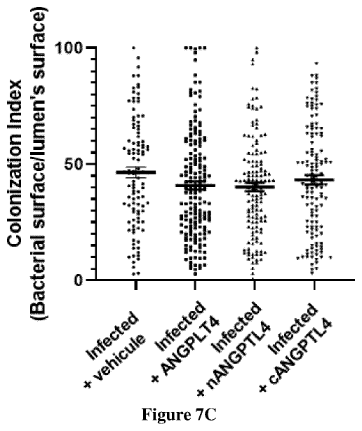

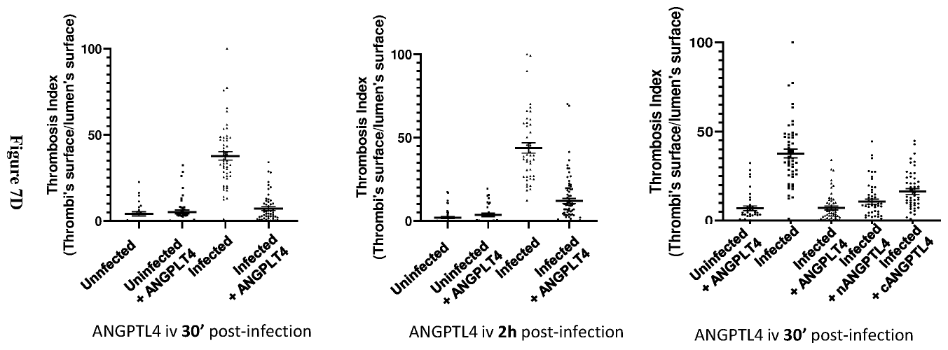

We therefore investigated the potential protective effect of ANGPTL4 in vivo on vascular alterations induced by N. meningitidis, using a validated and robust humanized mouse model of meningococcal infection, consisting of severe combined immunodeficiency (SCID) mice grafted with human skin that reproduce vascular lesions reminiscent of the purpuric lesions observed in patients [18, 19] (FIG. 7A). Following intravenous injection of 5×10⁶ bacteria, to mimic the conditions in patients with meningococcaemia (FIG. 7B), bacteria massively colonized the human dermal vessels contained within the skin grafts at 4 h post-infection (FIG. 7C). As previously described [20, 21], this vascular colonization was associated with a massive platelet and red blood cell aggregation, vessel occlusion (data not shown) and signs of compromised endothelial integrity, as assessed by the loss of the junction marker VE-cadherin (data not shown). Upon intravenous administration of recombinant human ANGPTL4 protein (1 μg/mouse), 30 min or 2 h after the initiation of infection (FIG. 7A), bacteraemia and vascular colonization at 4 hours post-infection were similar between mice treated with ANGPTL4 and control mice (FIG. 7B). However, ANGPTL4 treatment largely reduced vessel thrombosis (FIG. 7D) and preserved the integrity of the endothelial cell junctions, as assessed by the continuous staining of VE-cadherin (data not shown). In addition, whereas infection induced the release of pro-inflammatory factors (TNF-α, IL-6 and IL-8) in mice sera, along with biomarkers of vascular inflammation (soluble E-selectin, ICAM-1, VCAM-1) or vascular alteration (Angiopoetin-2, Thrombomodulin), ANGPTL4 treatment reduced serum levels of all these markers by 60-80% (data not shown). Administration of ANGPTL4 at 30 min or 2 h post-infection similarly reduced vascular lesions and thrombosis, whereas early administration was required to prevent the release of inflammatory factors, as no effect on these inflammatory markers was longer observed upon administration at 2 h (data not shown).

Interestingly, we noticed that both administration of the N-terminus and C-terminus domain of ANGPTL4 (1 μg/mouse) reduced vessel thrombosis induced by infection (FIG. 7D) with no effect on vascular colonization (FIG. 7C); while administration of nANGPTL4 preserved the integrity of the endothelial cell junctions (data not shown), administration of cANGPTL4 was most efficient at preventing the production of proinflammatory factors and biomarkers of vascular inflammation (data not shown), demonstrating a dual function of ANGPTL4 relying on its two distinct functional domains. As expected, no protective effects were observed on vascular alteration, thrombosis or the release of inflammatory markers upon treatment with ANGPTL3 or ANGPTL8 (data not shown), indicating that these effects were specific to ANGPTL4. These data demonstrate a clear protective effect of ANGPTL4 on the occurrence of vascular alteration, thrombosis and inflammation during meningoccaemia.

### ANGPTL4 Improves the Outcome of Meningococcal Infection

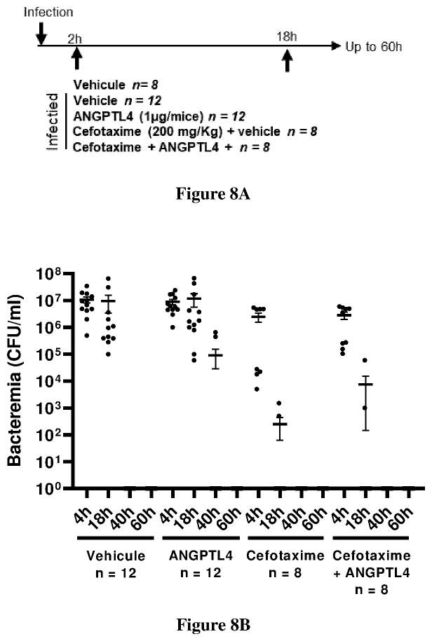

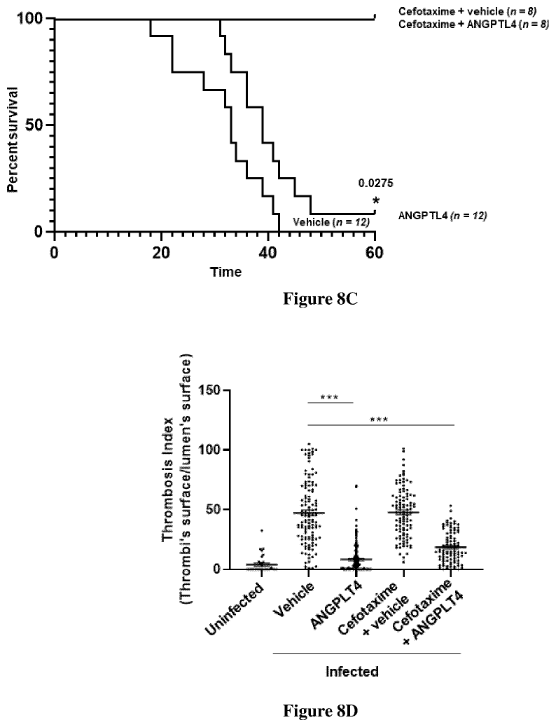

We then addressed whether ANGPTL4 improved the outcome of infected mice (FIG. 8A). In control mice, intravenous infection with 1×10⁶ bacteria led to a sustained bacteraemia at an average of 10⁶ to 5×10⁶ bacteria ml-1 at 18 h post-infection (FIG. 8B) and all mice died within two days (between 20 and 40 h post-infection) (FIG. 8C). Following intravenous administration of recombinant human ANGPTL4 (1 μg/mouse, i.v.), 2 h post-infection, while bacteraemia were similar to that of vehicle-treated control mice at 4 h and 18 h post-infection (FIG. 8B), the ANGPTL4-treated mice died at later time point (between 30 h and 50 h) and 15% survived to the infection (FIG. 8C). In contrast to infected control grafts, in which human dermal vessels were massively colonized, with intraluminal occlusive thrombi and presented signs of compromised endothelial integrity at the time of death (between 20 and 40 h post-infection), human dermal vessels from ANGPTL4-treated grafts had reduced signs of thrombosis and vascular leakage (data not shown).

We then analysed whether ANGPTL4 could provide a beneficial effect when administered with antibiotics. As previously observed [20], treatment with antibiotics (200 mg kg-1 cefotaxime) exerted a strong bactericidal effect (FIG. 8B) associated with mice survival (FIG. 8C), but it did not prevent the triggering of thrombosis at 4 h post-infection (FIG. 8D), nor vascular alterations associated with extravasation of red blood cells and massive infiltration of polymorphonuclear neutrophils at 72 h post-infection (data not shown). Upon treatment with antibiotics in combination with ANGPTL4, infected vessels presented signs of fibrinolysis associated with thrombi of reduced size, macrophage infiltration and preserved vascular integrity, the hallmarks of thrombus resolution (data not shown). Altogether these data indicate a beneficial effect of ANGPTL4 for restoring vessel patency and reducing the pathology and mortality associated with meningococcal infection. They also demonstrate the adjunctive effects of ANGPTL4 over antibiotic treatment by reducing the alteration of blood vessels.

### ANGPTL4 Protects Against Lethal Sepsis by Preventing Vascular Dysfunction and Organ Failure.

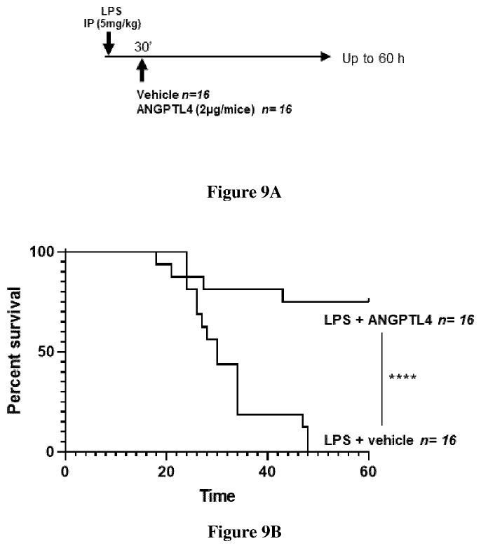

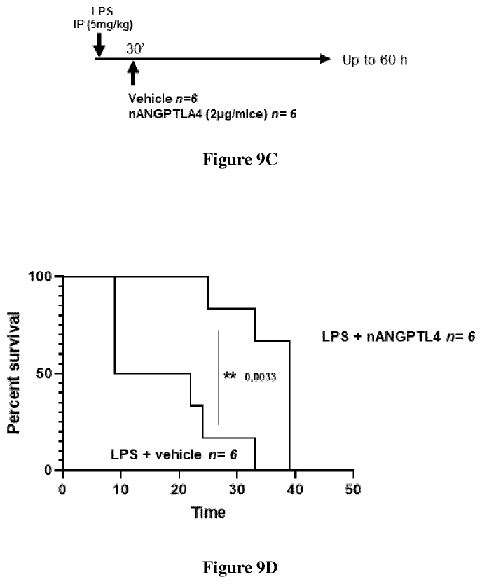

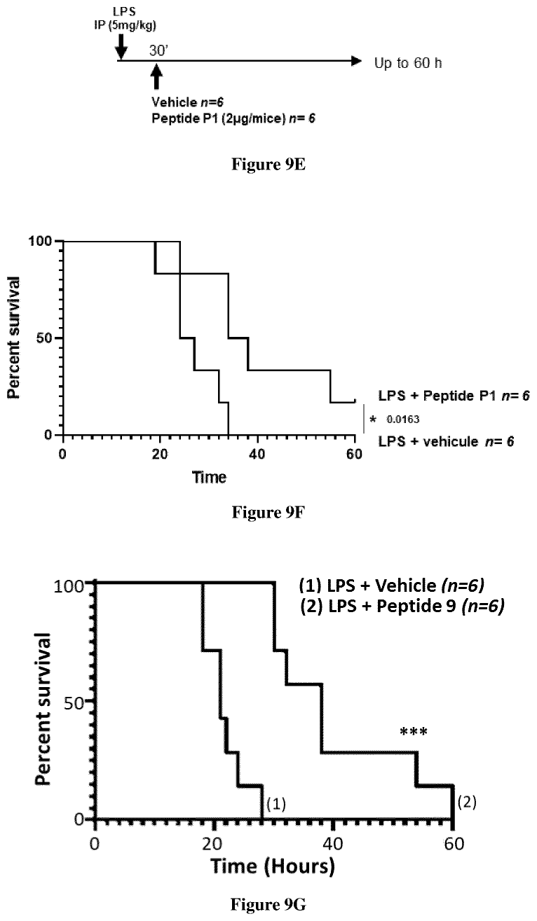

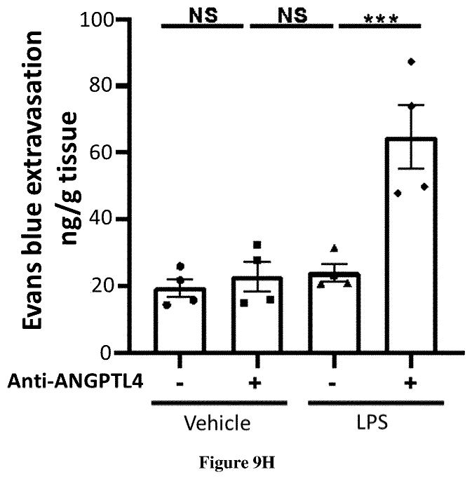

Since severe endothelial dysfunction, leading to dysregulation of hemostasis and vascular dysfunction, is central to the progression to organ failure during sepsis, we analysed whether ANGPTL4 could play a protective role in sepsis, using a lipopolysaccharide (LPS)-induced endotoxemia (FIG. 9A). After LPS-challenge (5 mg kg-1, i.p.), all mice died within two days (FIG. 9B). Administration of recombinant human ANGPTL4 (2 μg/mouse, i.v.) markedly reduced sepsis-associated mortality rate compared to the vehicle-treated control mice, since 80% survived to the infection (FIG. 9B). Overall weight loss was not significantly reduced in ANGPTL4-treated groups compared to vehicle-treated control groups, however the body temperature was rapidly stabilized in ANGPTL4-treated groups (data not shown).

We then evaluated the effect of ANGPTL4 on LPS-induced acute lung and kidney injury 18 h post-LPS challenge. LPS induced marked histopathological changes in the lung of vehicle-treated control mice with vessel wall thickening, collapsed alveolar sacs and intra-alveolar haemorrhage, while no pathological signs were observed in the lung of ANGPTL4-treated mice (data not shown). Similarly, while LPS induced profound renal haemorrhage in vehicle-treated control mice, no sign of haemorrhage was detected in ANGPTL4-treated mice (data not shown). These data demonstrate that ANGPTL4 confers efficient protection against LPS-induced acute lung and kidney injury by preventing severe endothelial dysfunctions and the progression to organ failure and death. As expected, administration of the nANGPTL4 (2 μg/mouse, i.v.) and of the derived peptide P1 or P9 reduced sepsis-associated mortality rate as efficiently as the full-length protein (FIGS. 9C-9F, 9G), while administration of cANGPTL4 or peptide P10 (2 μg/mouse, i.v.) did not significantly increased the survival rate (data not shown), further confirming that this effect relied on the ability of the N-terminus portion of ANGPTL4 to preserve vessel integrity. In sharp contrast, LPS challenge (4 mg kg-1, i.p.) did not compromise BBB integrity, as assessed by the lack of Evans blue extravasation in the brain of LPS-treated mice (FIG. 9H). Treatment with anti-ANGPTL4 blocking antibody (10 μg kg-1, i.v) together with LPS induced a marked Evans blue extravasation (FIG. 9H), indicating that endogenous production of ANGPTL4 at the CNS level protects against LPS-induced BBB disruption.

Collectively, these results demonstrate that ANGPTL4 is a major vascular stabilization factor conferring protection to bacterial infection. The use of human recombinant ANGPTL4 (or derived peptide) may reduce the incidence of vascular alteration, intravascular coagulation in bacterial diseases and reduce the heavy toll of sepsis.

### REFERENCES

Throughout this application, various references describe the state of the art to which this invention pertains. The disclosures of these references are hereby incorporated by reference into the present disclosure.
1 Cavaillon, J. M., M. Singer, and T. Skirecki, Sepsis therapies: learning from 30 years of failure of translational research to propose new leads. EMBO Mol Med, 2020. 12 (4): p. e10128.
2. Jarczak, D., S. Kluge, and A. Nierhaus, Sepsis-Pathophysiology and Therapeutic Concepts. Front Med (Lausanne), 2021. 8: p. 628302.
3. Angus, D. C. and T. van der Poll, Severe sepsis and septic shock. N Engl J Med, 2013. 369 (21): p. 2063.
4. Coureuil, M., et al., Invasive meningococcal disease: a disease of the endothelial cells. Trends Mol Med, 2014. 20 (10): p. 571-8.
5. Coureuil, M., et al., A journey into the brain: insight into how bacterial pathogens cross blood-brain barriers. Nat Rev Microbiol, 2017. 15 (3): p. 149-159.
6. Wolburg, H. and A. Lippoldt, Tight junctions of the blood-brain barrier:
   development, composition and regulation. Vascul Pharmacol, 2002. 38 (6): p. 323-37.
7. Abbott, N.J., et al., Structure and function of the blood-brain barrier. Neurobiol Dis, 2010. 37 (1): p. 13-25.
8 Daneman, R., et al., Pericytes are required for blood-brain barrier integrity during embryogenesis. Nature, 2010. 468 (7323): p. 562-6.
9. Abbott, N.J., L. Ronnback, and E. Hansson, Astrocyte-endothelial interactions at the blood-brain barrier. Nat Rev Neurosci, 2006. 7 (1): p. 41-53.
10. Caugant, D. A. and M. C. Maiden, Meningococcal carriage and disease—population biology and evolution. Vaccine, 2009. 27 Suppl 2: p. B64-70.
11. Mairey, E., et al., Cerebral microcirculation shear stress levels determine Neisseria meningitidis attachment sites along the blood-brain barrier. J Exp Med, 2006. 203 (8): p. 1939-50.
12. Pron, B., et al., Interaction of Neisseria maningitidis with the components of the blood-brain barrier correlates with an increased expression of PilC. J Infect Dis, 1997. 176 (5): p. 1285-92.
13. Yang, X., Y. Cheng, and G. Su, A review of the multifunctionality of angiopoietin-like 4 in eye disease. Biosci Rep, 2018. 38 (5).
14. Chong, H. C., et al., Angiopoietin-like 4 stimulates STAT3-mediated iNOS expression and enhances angiogenesis to accelerate wound healing in diabetic mice. Mol Ther, 2014. 22 (9): p. 1593-604.
15. Le Jan, S., et al., Angiopoietin-like 4 is a proangiogenic factor produced during ischemia and in conventional renal cell carcinoma. Am J Pathol, 2003. 162 (5): p. 1521-8.
16. Huang, R. L., et al., ANGPTL4 modulates vascular junction integrity by integrin signaling and disruption of intercellular VE-cadherin and claudin-5 clusters. Blood, 2011. 118 (14): p. 3990-4002.
17. Li, L., et al., Angiopoietin-like 4 Increases Pulmonary Tissue Leakiness and Damage during Influenza Pneumonia. Cell Rep, 2015. 10 (5): p. 654-663.
18. Zhang, B., et al., Protective effects of angiopoietin-like 4 on the blood-brain barrier in acute ischemic stroke treated with thrombolysis in mice. Neurosci Lett, 2017. 645: p. 113-120.
19. Galaup, A., et al., Angiopoietin-like 4 prevents metastasis through inhibition of vascular permeability and tumor cell motility and invasiveness. Proc Natl Acad Sci USA, 2006. 103 (49): p. 18721-6.
20. Bouleti, C., et al., Protective effects of angiopoietin-like 4 on cerebrovascular and functional damages in ischaemic stroke. Eur Heart J, 2013. 34 (47): p. 3657-68.

1. A peptide derived from the protein ANGPTL4 comprising or consisting of the amino acid sequence X₁SALERRLSACGSX₁₄X₁₅ or a function-conservative variant thereof, wherein X₁ is no amino acid or leucine (L), X₁₄ is no amino acid or alanine (A) and X₁₅ is no amino acid or cysteine (C).

2. A peptide derived from the protein ANGPTL4 according to claim 1, wherein the peptide comprises or consists of the amino acid residue at position 66 to the amino acid residue at position 80 in SEQ ID NO:1 wherein the amino acid at position 76 and at position 80 is a naturally occurring amino acid or a function-conservative variant thereof.

3. A peptide derived from the protein ANGPTL4 according to claim 2 wherein the peptide comprises or consists of the amino acid residue at position 65 to the amino acid residue at position 80 in SEQ ID NO: 1 wherein the amino acid at position 76 and at position 80 is a naturally occurring amino acid or a function-conservative variant thereof.

4. A peptide derived from the protein ANGPTL4 according to claim 2 wherein the peptide comprises or consists of the amino acid residue at position 56 to the amino acid residue at position 83 in SEQ ID NO: 1 wherein the amino acid at position 76 and at position 80 are any is a naturally occurring amino acid (peptide P4 or peptide P4′) or a function-conservative variant thereof.

5. A peptide derived from the protein ANGPTL4 according to claim 2 wherein the peptide comprises or consists of the amino acid residue at position 38 to the amino acid residue at position 83 in SEQ ID NO: 1 wherein the amino acid at position 76 and at position 80 is a naturally occurring amino acid or a function-conservative variant thereof.

6. A peptide derived from the protein ANGPTL4 according to claim 2 wherein the peptide comprises or consists of the amino acid residue at position 26 to the amino acid residue at position 164 in SEQ ID NO: 1 wherein the amino acid at position 76 and at position 80 is a naturally occurring amino acid or a function-conservative variant thereof.

7. A peptide derived from the protein ANGPTL4 according to the claim 2 wherein the peptide comprises or consists of the amino acid residue at position 1 to the amino acid residue at position 406 in SEQ ID NO:1 wherein the amino acid at position 76 and at position 80 is a naturally occurring amino acid or a function-conservative variant thereof.

8. A peptide derived from the protein ANGPTL4 according to claim 1, wherein the amino acid at position 76 and at position 80 is an amino acid selected from the group consisting of cysteine, or alanine and serine.

9. A peptide derived from the protein ANGPTL4 according to claim 1, wherein the peptide consists of the amino acids sequence as set forth in SEQ ID NO: 1, SEQ ID NOS: 8 to 30 or SEQ ID NOS: 37 to 64.

10. A nucleic acid sequence encoding a peptide according to claim 1.

11. A method of treating and infectious disease in a subject in need thereof, comprising administering to the subject a therapeutically effective amount of a peptide according to claim 1 or a nucleic acid encoding the peptide.

12. The method according to claim 11 wherein the infectious disease is a bacterial infection.

13. The method according to claim 12 wherein the bacterial infection is sepsis due to the bacterial infection.

14. A method for treating an infectious disease in a subject in need thereof comprising administering to a subject a therapeutically effective amount of the peptide according to claim 1, a protein comprising the peptide, a nucleic acid encoding the peptide, or a vector or host cell comprising the nucleic acid.

15. The method of claim 14, further comprising administering to the subject, simultaneously, separately or sequentially, at least one additional anti-pathogen infection agent.

16. A therapeutic composition comprising an antibody or a peptide according to claim 1, a protein comprising the peptide, a nucleic acid encoding the peptide, or a vector or host cell comprising the nucleic acid.


SEQUENCE LISTING (65 sequences)

SEQ ID NO: 1 - protein, 406 aa, Homo sapiens
MSGAPTAGAALMLCAATAVLLSAQGGPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 2 - protein, 15 aa, synthetic construct
  REGION 1..15: note = peptide P9 or peptide P9'
  VARIANT 11: note = X is any naturally occurring amino acid
  VARIANT 15: note = X is any naturally occurring amino acid
LSALERRLSAXGSAX

SEQ ID NO: 3 - protein, 16 aa, synthetic construct
  REGION 1..16: note = peptide P6 or peptide P6'
  VARIANT 12: note = X is any naturally occurring amino acid
  VARIANT 16: note = X is any naturally occurring amino acid
QLSALERRLSAXGSAX

SEQ ID NO: 4 - protein, 28 aa, synthetic construct
  REGION 1..28: note = peptide P4 or peptide P4'
  VARIANT 21: note = X is any naturally occurring amino acid
  VARIANT 25: note = X is any naturally occurring amino acid
REHAERTRSQLSALERRLSAXGSAXQGT

SEQ ID NO: 5 - protein, 46 aa, synthetic construct
  REGION 1..46: note = Peptide P1 or P1'
  VARIANT 39: note = X is any naturally occurring amino acid
  VARIANT 43: note = X is any naturally occurring amino acid
WDEMNVLAHGLLQLGQGLREHAERTRSQLSALERRLSAXGSAXQGT

SEQ ID NO: 6 - protein, 138 aa, synthetic construct
  REGION 1..138: note = nANGPTL4 or nANGPTL4'
  VARIANT 51: note = X is any naturally occurring amino acid
  VARIANT 55: note = X is any naturally occurring amino acid
GPVQSKSPRFASWDEMNVLAHGLLQLGQGLREHAERTRSQLSALERRLSAXGSAXQGTEGSTDLPLAPESRVDPEVLHSLQTQLKAQNSRIQQLFHKVAQQQRHLEKQHLRIQHLQSQFGLLDHKHLDHEVAKPARRK

SEQ ID NO: 7 - protein, 406 aa, synthetic construct
  REGION 1..406: note = ANGPTL4 or ANGPTL4'
  VARIANT 80: note = X is any naturally occurring amino acid
  VARIANT 76: note = X is any naturally occurring amino acid
MSGAPTAGAALMLCAATAVLLSAQGGPVQSKSPRFASWDEMNVLAHGLLQLGQGLREHAERTRSQLSALERRLSAXGSAXQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 8 - protein, 15 aa, synthetic construct
  REGION 1..15: note = Peptide P9
LSALERRLSACGSAC

SEQ ID NO: 9 - protein, 15 aa, synthetic construct
  REGION 1..15: note = Peptide P9 C76A, C80A
LSALERRLSAAGSAA

SEQ ID NO: 10 - protein, 15 aa, synthetic construct
  REGION 1..15: note = Peptide P9 C76A
LSALERRLSAAGSAC

SEQ ID NO: 11 - protein, 15 aa, synthetic construct
  REGION 1..15: note = Peptide P9 C80A
LSALERRLSACGSAA

SEQ ID NO: 12 - protein, 16 aa, synthetic construct
  REGION 1..16: note = Peptide P6
QLSALERRLSACGSAC

SEQ ID NO: 13 - protein, 16 aa, synthetic construct
  REGION 1..16: note = Peptide P6 C76A, C80A
QLSALERRLSAAGSAA

SEQ ID NO: 14 - protein, 16 aa, synthetic construct
  REGION 1..16: note = Peptide P6: C76A
QLSALERRLSAAGSAC

SEQ ID NO: 15 - protein, 16 aa, synthetic construct
  REGION 1..16: note = Peptide P6 C80A
QLSALERRLSACGSAA

SEQ ID NO: 16 - protein, 28 aa, synthetic construct
  REGION 1..28: note = Peptide P4
REHAERTRSQLSALERRLSACGSACQGT

SEQ ID NO: 17 - protein, 28 aa, synthetic construct
  REGION 1..28: note = Peptide P4 C76A, C80A
REHAERTRSQLSALERRLSAAGSAAQGT

SEQ ID NO: 18 - protein, 28 aa, synthetic construct
  REGION 1..28: note = Peptide P4 C76A
REHAERTRSQLSALERRLSAAGSACQGT

SEQ ID NO: 19 - protein, 28 aa, synthetic construct
  REGION 1..28: note = Peptide P4 C80A
REHAERTRSQLSALERRLSACGSAAQGT

SEQ ID NO: 20 - protein, 46 aa, synthetic construct
  REGION 1..46: note = Peptide P1
WDEMNVLAHGLLQLGQGLREHAERTRSQLSALERRLSACGSACQGT

SEQ ID NO: 21 - protein, 46 aa, synthetic construct
  REGION 1..46: note = Peptide P1 C76A, C80A
WDEMNVLAHGLLQLGQGLREHAERTRSQLSALERRLSAAGSAAQGT

SEQ ID NO: 22 - protein, 46 aa, synthetic construct
  REGION 1..46: note = Peptide P1 C76A
WDEMNVLAHGLLQLGQGLREHAERTRSQLSALERRLSAAGSACQGT

SEQ ID NO: 23 - protein, 46 aa, synthetic construct
  REGION 1..46: note = Peptide P1 C80A
WDEMNVLAHGLLQLGQGLREHAERTRSQLSALERRLSACGSAAQGT

SEQ ID NO: 24 - protein, 138 aa, synthetic construct
  REGION 1..138: note = nANGPTL4
GPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRK

SEQ ID NO: 25 - protein, 138 aa, synthetic construct
  REGION 1..138: note = Peptide nANGPTL4 C76A, C80A
GPVQSKSPRFASWDEMNVLAHGLLQLGQGLREHAERTRSQLSALERRLSAAGSAAQGTEGSTDLPLAPESRVDPEVLHSLQTQLKAQNSRIQQLFHKVAQQQRHLEKQHLRIQHLQSQFGLLDHKHLDHEVAKPARRK

SEQ ID NO: 26 - protein, 138 aa, synthetic construct
  REGION 1..138: note = Peptide nANGPTL4 C76A
GPVQSKSPRFASWDEMNVLAHGLLQLGQGLREHAERTRSQLSALERRLSAAGSACQGTEGSTDLPLAPESRVDPEVLHSLQTQLKAQNSRIQQLFHKVAQQQRHLEKQHLRIQHLQSQFGLLDHKHLDHEVAKPARRK

SEQ ID NO: 27 - protein, 138 aa, synthetic construct
  REGION 1..138: note = Peptide nANGPTL4 C80A
GPVQSKSPRFASWDEMNVLAHGLLQLGQGLREHAERTRSQLSALERRLSACGSAAQGTEGSTDLPLAPESRVDPEVLHSLQTQLKAQNSRIQQLFHKVAQQQRHLEKQHLRIQHLQSQFGLLDHKHLDHEVAKPARRK

SEQ ID NO: 28 - protein, 406 aa, synthetic construct
  REGION 1..406: note = Protein ANGPTL4 C76A
MSGAPTAGAALMLCAATAVLLSAQGGPVQSKSPRFASWDEMNVLAHGLLQLGQGLREHAERTRSQLSALERRLSAAGSAAQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 29 - protein, 406 aa, synthetic construct
  REGION 1..406: note = Protein ANGPTL4 C76A
MSGAPTAGAALMLCAATAVLLSAQGGPVQSKSPRFASWDEMNVLAHGLLQLGQGLREHAERTRSQLSALERRLSAAGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 30 - protein, 406 aa, synthetic construct
  REGION 1..406: note = Protein ANGPTL4 C80A
MSGAPTAGAALMLCAATAVLLSAQGGPVQSKSPRFASWDEMNVLAHGLLQLGQGLREHAERTRSQLSALERRLSACGSAAQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 31 - protein, 50 aa, synthetic construct
  REGION 1..50: note = Peptide P2
PEVLHSLQTQLKAQNSRIQQLFHKVAQQQRHLEKQHLRIQHLQSQFGLLD

SEQ ID NO: 32 - protein, 18 aa, synthetic construct
  REGION 1..18: note = Peptide P3
WDEMNVLAHGLLQLGQGL

SEQ ID NO: 33 - protein, 18 aa, synthetic construct
  REGION 1..18: note = Peptide P5
REHAERTRSQLSALERRL

SEQ ID NO: 34 - protein, 15 aa, synthetic construct
  REGION 1..15: note = Peptide P7
RTRSQLSALERRLSA

SEQ ID NO: 35 - protein, 10 aa, synthetic construct
  REGION 1..10: note = Peptide P8
LSALERRLSA

SEQ ID NO: 36 - protein, 12 aa, synthetic construct
  REGION 1..12: note = Peptide P10
SALERRLSACGS

SEQ ID NO: 37 - protein, 15 aa, synthetic construct
LSALERRLSASGSAS

SEQ ID NO: 38 - protein, 15 aa, synthetic construct
LSALERRLSASGSAC

SEQ ID NO: 39 - protein, 15 aa, synthetic construct
LSALERRLSACGSAS

SEQ ID NO: 40 - protein, 16 aa, synthetic construct
QLSALERRLSASGSAS

SEQ ID NO: 41 - protein, 16 aa, synthetic construct
QLSALERRLSASGSAC

SEQ ID NO: 42 - protein, 16 aa, synthetic construct
QLSALERRLSACGSAS

SEQ ID NO: 43 - protein, 28 aa, synthetic construct
REHAERTRSQLSALERRLSASGSASQGT

SEQ ID NO: 44 - protein, 28 aa, synthetic construct
REHAERTRSQLSALERRLSASGSACQGT

SEQ ID NO: 45 - protein, 28 aa, synthetic construct
REHAERTRSQLSALERRLSACGSASQGT

SEQ ID NO: 46 - protein, 46 aa, synthetic construct
WDEMNVLAHGLLQLGQGLREHAERTRSQLSALERRLSASGSASQGT

SEQ ID NO: 47 - protein, 46 aa, synthetic construct
WDEMNVLAHGLLQLGQGLREHAERTRSQLSALERRLSASGSACQGT

SEQ ID NO: 48 - protein, 46 aa, synthetic construct
WDEMNVLAHGLLQLGQGLREHAERTRSQLSALERRLSACGSASQGT

SEQ ID NO: 49 - protein, 138 aa, synthetic construct
GPVQSKSPRFASWDEMNVLAHGLLQLGQGLREHAERTRSQLSALERRLSASGSASQGTEGSTDLPLAPESRVDPEVLHSLQTQLKAQNSRIQQLFHKVAQQQRHLEKQHLRIQHLQSQFGLLDHKHLDHEVAKPARRK

SEQ ID NO: 50 - protein, 138 aa, synthetic construct
GPVQSKSPRFASWDEMNVLAHGLLQLGQGLREHAERTRSQLSALERRLSASGSACQGTEGSTDLPLAPESRVDPEVLHSLQTQLKAQNSRIQQLFHKVAQQQRHLEKQHLRIQHLQSQFGLLDHKHLDHEVAKPARRK

SEQ ID NO: 51 - protein, 138 aa, synthetic construct
GPVQSKSPRFASWDEMNVLAHGLLQLGQGLREHAERTRSQLSALERRLSACGSASQGTEGSTDLPLAPESRVDPEVLHSLQTQLKAQNSRIQQLFHKVAQQQRHLEKQHLRIQHLQSQFGLLDHKHLDHEVAKPARRK

SEQ ID NO: 52 - protein, 406 aa, synthetic construct
MSGAPTAGAALMLCAATAVLLSAQGGPVQSKSPRFASWDEMNVLAHGLLQLGQGLREHAERTRSQLSALERRLSASGSASQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 53 - protein, 406 aa, synthetic construct
MSGAPTAGAALMLCAATAVLLSAQGGPVQSKSPRFASWDEMNVLAHGLLQLGQGLREHAERTRSQLSALERRLSASGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 54 - protein, 406 aa, synthetic construct
MSGAPTAGAALMLCAATAVLLSAQGGPVQSKSPRFASWDEMNVLAHGLLQLGQGLREHAERTRSQLSALERRLSACGSASQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 55 - protein, 13 aa, synthetic construct
LSALERRLSACGS

SEQ ID NO: 56 - protein, 13 aa, synthetic construct
LSALERRLSAAGS

SEQ ID NO: 57 - protein, 13 aa, synthetic construct
LSALERRLSASGS

SEQ ID NO: 58 - protein, 14 aa, synthetic construct
SALERRLSACGSAC

SEQ ID NO: 59 - protein, 14 aa, synthetic construct
SALERRLSAAGSAA

SEQ ID NO: 60 - protein, 14 aa, synthetic construct
SALERRLSAAGSAC

SEQ ID NO: 61 - protein, 14 aa, synthetic construct
SALERRLSACGSAA

SEQ ID NO: 62 - protein, 14 aa, synthetic construct
SALERRLSASGSAS

SEQ ID NO: 63 - protein, 14 aa, synthetic construct
SALERRLSASGSAC

SEQ ID NO: 64 - protein, 14 aa, synthetic construct
SALERRLSACGSAS

SEQ ID NO: 65 - protein, 15 aa, synthetic construct
  VARIANT 1: note = X is no amino acid or leucine
  VARIANT 14: note = X is no amino acid or alanine
  VARIANT 15: note = X is no amino acid or cysteine
XSALERRLSACGSXX